SEQUENCE LISTING (24 sequences)

SEQ ID NO: 1 - protein, 755 aa, Homo sapiens
MARKLSVILILTFALSVTNPLHELKAAAFPQTTEKISPNWESGINVDLAISTRQYHLQQLFYRYGENNSLSVEGFRKLLQNIGIDKIKRIHIHHDHDHHSDHEHHSDHERHSDHEHHSEHEHHSDHDHHSHHNHAASGKNKRKALCPDHDSDSSGKDPRNSQGKGAHRPEHASGRRNVKDSVSASEVTSTVYNTVSEGTHFLETIETPRPGKLFPKDVSSSTPPSVTSKSRVSRLAGRKTNESVSEPRKGFMYSRNTNENPQECFNASKLLTSHGMGIQVPLNATEFNYLCPAIINQIDARSCLIHTSEKKAEIPPKTYSLQIAWVGGFIAISIISFLSLLGVILVPLMNRVFFKFLLSFLVALAVGTLSGDAFLHLLPHSHASHHHSHSHEEPAMEMKRGPLFSHLSSQNIEESAYFDSTWKGLTALGGLYFMFLVEHVLTLIKQFKDKKKKNQKKPENDDDVEIKKQLSKYESQLSTNEEKVDTDDRTEGYLRADSQEPSHFDSQQPAVLEEEEVMIAHAHPQEVYNEYVPRGCKNKCHSHFHDTLGQSDDLIHHHHDYHHILHHHHHQNHHPHSHSQRYSREELKDAGVATLAWMVIMGDGLHNFSDGLAIGAAFTEGLSSGLSTSVAVFCHELPHELGDFAVLLKAGMTVKQAVLYNALSAMLAYLGMATGIFIGHYAENVSMWIFALTAGLFMYVALVDMVPEMLHNDASDHGCSRWGYFFLQNAGMLLGFGIMLLISIFEHKIVFRINF

SEQ ID NO: 2 - protein, 749 aa, Homo sapiens
MARKLSVILILTFALSVTNPLHELKAAAFPQTTEKISPNWESGINVDLAISTRQYHLQQLFYRYGENNSLSVEGFRKLLQNIGIDKIKRIHIHHDHDHHSDHEHHSDHERHSDHEHHSDHEHHSDHNHAASGKNKRKALCPDHDSDSSGKDPRNSQGKGAHRPEHASGRRNVKDSVSASEVTSTVYNTVSEGTHFLETIETPRPGKLFPKDVSSSTPPSVTSKSRVSRLAGRKTNESVSEPRKGFMYSRNTNENPQECFNASKLLTSHGMGIQVPLNATEFNYLCPAIINQIDARSCLIHTSEKKAEIPPKTYSLQIAWVGGFIAISIISFLSLLGVILVPLMNRVFFKFLLSFLVALAVGTLSGDAFLHLLPHSHASHHHSHSHEEPAMEMKRGPLFSHLSSQNIEESAYFDSTWKGLTALGGLYFMFLVEHVLTLIKQFKDKKKKNQKKPENDDDVEIKKQLSKYESQLSTNEEKVDTDDRTEGYLRADSQEPSHFDSQQPAVLEEEEVMIAHAHPQEVYNEYVPRGCKNKCHSHFHDTLGQSDDLIHHHHDYHHILHHHHHQNHHPHSHSQRYSREELKDAGVATLAWMVIMGDGLHNFSDGLAIGAAFTEGLSSGLSTSVAVFCHELPHELGDFAVLLKAGMTVKQAVLYNALSAMLAYLGMATGIFIGHYAENVSMWIFALTAGLFMYVALVDMVPEMLHNDASDHGCSRWGYFFLQNAGMLLGFGIMLLISIFEHKIVFRINF

SEQ ID NO: 3 - protein, 113 aa, Artificial Sequence
  Light chain variable region of hLIV22
DVVMTQSPLSLPVTLGQPASISCRSSQSLLHSSGNTYLEWYQQRPGQSPRPLIYKISTRFSGVPDRFSGSGSGTDFTLKISRVEAEDVGVYYCFQGSHVPYTFGGGTKVEIKR

SEQ ID NO: 4 - protein, 120 aa, Artificial Sequence
  Heavy chain variable region of hLIV22
QVQLVQSGAEVKKPGASVKVSCKASGLTIEDYYMHWVRQAPGQGLEWMGWIDPENGDTEYGPKFQGRVTMTRDTSINTAYMELSRLRSDDTAVYYCAVHNAHYGTWFAYWGQGTLVTVSS

SEQ ID NO: 5 - protein, 15 aa, Homo Sapiens
RASKGVSTSGYSYLH

SEQ ID NO: 6 - protein, 7 aa, Homo Sapiens
LASYLES

SEQ ID NO: 7 - protein, 9 aa, Homo Sapiens
QHSRDLPLT

SEQ ID NO: 8 - protein, 5 aa, Homo Sapiens
NYYMY

SEQ ID NO: 9 - protein, 17 aa, Homo Sapiens
GINPSNGGTNFNEKFKN

SEQ ID NO: 10 - protein, 11 aa, Homo Sapiens
RDYRFDMGFDY

SEQ ID NO: 11 - protein, 15 aa, Homo Sapiens
RASKSVSTSGFSYLH

SEQ ID NO: 12 - protein, 7 aa, Homo Sapiens
LASNLES

SEQ ID NO: 13 - protein, 9 aa, Homo Sapiens
QHSWELPLT

SEQ ID NO: 14 - protein, 5 aa, Homo Sapiens
SYYLY

SEQ ID NO: 15 - protein, 17 aa, Homo Sapiens
GVNPSNGGTNFSEKFKS

SEQ ID NO: 16 - protein, 11 aa, Homo Sapiens
RDSNYDGGFDY

SEQ ID NO: 17 - protein, 120 aa, Homo Sapiens [sequence omitted]

SEQ ID NO: 18 - protein, 111 aa, Homo Sapiens [sequence omitted]

SEQ ID NO: 19 - protein, 111 aa, Homo Sapiens
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIK

SEQ ID NO: 20 - protein, 111 aa, Homo Sapiens
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIK

SEQ ID NO: 21 - protein, 447 aa, Homo Sapiens
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 218 aa, Homo Sapiens
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 218 aa, Homo Sapiens
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 218 aa, Homo Sapiens
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2021/0228676 A1
(43) Pub. Date: Jul. 29, 2021

(54) Combination Therapy With LIV1-ADC and PD-1 Antagonist

(71) Applicant: SEAGEN INC., Bothell, WA (US)

(72) Inventors: Zejing Wang, Bellevue, WA (US); Nicholas Choong, Belmont, CA (US); Django Sussman, Seattle, WA (US)

(21) Appl. No.: 17/116,543

(22) Filed: Dec. 9, 2020

Related U.S. Application Data
(60) Provisional application No. 63/110,692, filed on Nov. 6, 2020.
(60) Provisional application No. 63/076,664, filed on Sep. 10, 2020.
(60) Provisional application No. 63/031,528, filed on May 28, 2020.
(60) Provisional application No. 62/945,885, filed on Dec. 9, 2019.

(51) Int. Cl. A61K 38/07; A61K 47/68; A61K 39/395; A61K 47/65; A61P 35/00

(52) U.S. Cl. CPC A61K 38/07; A61K 47/6849; A61K 47/6817; A61K 2039/545; A61K 47/65; A61P 35/00; A61K 39/3955

(57) ABSTRACT

The present disclosure relates, in general, to methods for treating LIV-1-expressing cancers comprising administering an anti-LIV-1 antibody drug conjugate (LIV-1-ADC) in combination with a PD-1 antagonist, such as an anti-PD-1 antibody.

### CROSS REFERENCE TO RELATED APPLICATIONS

The present application claims the priority benefit of U.S. Provisional patent Application No. 62/945,885, filed Dec. 9, 2019, U.S. Provisional Patent Application No. 63/031,528, filed May 28, 2020, U.S. Provisional Patent Application No. 63/076,664, filed Sep. 10, 2020, and U.S. Provisional Patent Application No. 63/110,692, filed Nov. 6, 2020, incorporated by reference herein in their entireties.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “55109_Seqlisting.txt”, which was created on Dec. 7, 2020 and is 31,310 bytes in size. The subject matter of the Sequence Listing is incorporated herein in its entirety by reference.

### FIELD OF THE DISCLOSURE

This disclosure relates, in general, to methods for the treatment of cancer comprising administering a LIV-1-ADC and a PD-1 antagonist, such as an anti-PD-1 antibody.

### BACKGROUND

LIV-1 is a member of the LZT (LIV-1-ZIP Zinc Transporters) subfamily of zinc transporter proteins. Taylor et al., Biochim. Biophys. Acta 1611:16-30 (2003). Computer analysis of the LIV-1 protein reveals a potential metalloprotease motif, fitting the consensus sequence for the catalytic zinc-binding site motif of the zinc metalloprotease. LIV-1 mRNA is primarily expressed in breast, prostate, pituitary gland and brain tissue.

The LIV-1 protein has also been implicated in certain cancerous conditions, e.g., breast cancer and prostate cancer. The detection of LIV-1 is associated with estrogen receptor-positive breast cancer, McClelland et al., Br. J. Cancer 77:1653-1656 (1998), and the metastatic spread of these cancers to the regional lymph nodes. Manning et al., Eur. J. Cancer 30A:675-678 (1994).

SGN-LIV1A is a LIV-1-directed antibody-drug conjugate (ADC) consisting of three components: 1) the humanized antibody hLIV22, specific for human LIV-1, 2) the microtubule disrupting agent monomethyl auristatin E (MMAE), and 3) a stable linker, valine-citrulline (vc), that covalently attaches MMAE to hLIV22. The proposed mechanism of action (MOA) is imitated by SGN-LIV1A binding to LIV-1 on the cell surface followed by internalization of the ADC. Upon trafficking to lysosomes, the delivered drug (MMAE) is released through proteolytic degradation of the vc linker. Binding of the released drug to tubulin disrupts the microtubule network, leading to cell cycle arrest and apoptosis. SGN-LIV1A is also known as Ladiratuzumab Vedotin (LV). Treatment with ladiratuzumab vedotin, is associated with mitotic arrest, infiltration of macrophages, and upregulation of cytokine signaling (Specht et al., Ann Oncol. 2018; 29(Suppl 8):viii92).

SGN-LIV1A has been shown to reduce tumor volumes in vivo, and is currently being evaluated in a phase 1 clinical trial for patients with LIV-1-positive metastatic breast cancer. Preclinical reports have demonstrated that treatment with LV monotherapy induces immunogenic cell death (ICD) (Schmid et al., N Engl J Med. 2018; 379(22): 2108-21).

### SUMMARY

The present disclosure provides methods for treating cancer, in particular breast cancer, comprising administering an anti-LIV1 antibody drug conjugate in combination with a PD-1 antagonist to treat cancer, e.g., a LIV-1 expressing or checkpoint protein expressing cancer.

In various embodiments, the disclosure provides a method for treating a subject having or at risk of cancer, the method comprising administering to the subject a LIV-1 antibody drug conjugate (LIV-1-ADC) and a PD-1 antagonist selected from the group consisting of an anti-PD-1 antibody or an anti-PD-L1 antibody.

In various embodiments, the subject has breast cancer. In one embodiment, the breast cancer is triple negative breast cancer, triple positive breast cancer, HER2-positive breast cancer, or hormone receptor positive cancer. In one embodiment, the subject has triple negative breast cancer. In various embodiments, the subject has unresectable locally-advanced or metastatic (LA/M) triple negative breast cancer (TNBC).

In various embodiments, the disclosure provides a method for treating a subject having or at risk of triple negative breast cancer, the method comprising administering to the subject a LIV-1 antibody drug conjugate (LIV-1-ADC) and a PD-1 antagonist, wherein the PD-1 antagonist is an anti-PD-1 antibody or an anti-PD-L1 antibody.

Further contemplated is a method for treating a subject having de novo metastatic triple negative breast cancer, the method comprising administering to the subject a LIV-1 antibody drug conjugate (LIV-1-ADC) and a PD-1 antagonist, wherein the PD-1 antagonist is an anti-PD-1 antibody or an anti-PD-L1 antibody.

In various embodiments, the subject has prostate cancer, ovarian cancer, endrometrial cancer, pancreatic cancer, lung cancer, a cervical cancer, a melanoma, or squamous cell carcinoma.

In various embodiments, the subject has not previously received cytotoxic therapy.

In various embodiments, the LIV-1-ADC is administered at a dosage between 1.0 mg/kg and 4 mg/kg of the subject's body weight. In one embodiment, the LIV-1-ADC is administered at a dosage of 1.0 mg/kg of the subject's body weight. In one embodiment, the LIV-1-ADC is administered at a dosage of 1.25 mg/kg of the subject's body weight. In one embodiment, the LIV-1-ADC is administered at a dosage of 1.5 mg/kg of the subject's body weight. In one embodiment, the LIV-1-ADC is administered at a dosage of 1.75 mg/kg of the subject's body weight. In one embodiment, the LIV-1-ADC is administered at a dosage of 2.0 mg/kg of the subject's body weight. In one embodiment, the LIV-1-ADC is administered at a dosage of 2.5 mg/kg of the subject's body weight.

In various embodiments, the LIV-1-ADC is administered once weekly. In various embodiments, the LIV-1-ADC is administered once every 3 weeks. In various embodiments, the LIV-1-ADC is administered by intravenous injection. In various embodiments, the LIV-1-ADC is administered by intravenous infusion. In various embodiments, the LIV1-ADC and PD-1 antagonist therapy is administered for at least 3 cycles, and up to 6, 8, or 10 cycles, for example from 3 to 6 cycles, or 3 to 8 cycles, or for 3, 4, 5, 6, 7, 8, 9 or 10 cycles. In various embodiments, the cycle is a three week cycle.

In various embodiments, the PD-1 antagonist is an anti-PD-1 antibody selected from the group consisting of pembrolizumab or nivolumab. In one embodiment, the PD-1 antagonist is the anti-PD-1 antibody pembrolizumab. In various embodiments, the pembrolizumab is administered at a dosage between 100 and 300 mg, e.g., once every three weeks.

In various embodiments, the PD-1 antagonist is administered by intravenous infusion. In various embodiments, the anti-PD-1 antibody is administered every three weeks by intravenous infusion.

In various embodiments, the anti-LIV-1 antibody of the LIV-1-ADC is a monoclonal anti-LIV-1 antibody. In various embodiments, the anti-LIV-1 antibody of the LIV-1-ADC comprises a humanized hLIV22 antibody.

In various embodiments, the anti-LIV-1 antibody of the LIV-1-ADC comprises i) a heavy chain CDR1, CDR2, and CDR3 of the hLIV22 antibody and ii) a light chain CDR1, CDR2, and CDR3 of the hLIV22 antibody.

In various embodiments, the anti-LIV-1 antibody of the LIV-1-ADC comprises i) an amino acid sequence at least 85% identical to a heavy chain variable region set out in SEQ ID NO: 4 and ii) an amino acid sequence at least 85% identical to a light chain variable region set out in SEQ ID NO: 3. In various embodiments, the anti-LIV-1 antibody of the LIV-1-ADC comprises i) an amino acid sequence at least 90% identical to a heavy chain variable region set out in SEQ ID NO: 4 and ii) an amino acid sequence at least 90% identical to a light chain variable region set out in SEQ ID NO: 3. In various embodiments, the anti-LIV-1 antibody of the LIV-1-ADC comprises i) an amino acid sequence at least 95% identical to a heavy chain variable region set out in SEQ ID NO: 4 and ii) an amino acid sequence at least 95% identical to a light chain variable region set out in SEQ ID NO: 3. In various embodiments, the anti-LIV-1 antibody of the LIV-1-ADC comprises i) a heavy chain variable region amino acid sequence set out in SEQ ID NO: 4 and ii) a light chain variable region amino acid sequence set out in SEQ ID NO: 3. It is contemplated that variable region variant antibodies retain the heavy and light chain CDR sequences of the parental antibody.

In various embodiments, the antibody drug conjugate comprises monomethyl auristatin E and a protease-cleavable linker. In various embodiments, the protease cleavable linker comprises a thiolreactive spacer and a dipeptide. In various embodiments, the protease cleavable linker consists of a thiolreactive maleimidocaproyl spacer, a valine-citrulline dipeptide, and a p-amino-benzyloxycarbonyl spacer.

In various embodiments, the LIV-1-ADC is ladiratuzumab vedotin.

In various embodiments, (i) the anti-PD-1 antibody cross-competes with nivolumab or pembrolizumab for binding to human PD-1; (ii) the anti-PD-1 antibody binds to the same epitope as nivolumab or pembrolizumab; (iii) the anti-PD-1 antibody is nivolumab; or (iv) the anti-PD-1 antibody is pembrolizumab.

In various embodiments, (i) the anti-PD-1 antibody cross-competes with nivolumab or pembrolizumab for binding to human PD-1; (ii) the anti-PD-1 antibody binds to the same epitope as nivolumab or pembrolizumab; (iii) the anti-PD-1 antibody is nivolumab; (iv) the anti-PD-1 antibody is pembrolizumab; or (v) the anti-PD-1 antibody is a pembrolizumab variant.

In certain embodiments, the anti-PD-1 antibody is pembrolizumab or nivolumab.

In one embodiment, the anti-PD-1 antibody is pembrolizumab. In various embodiments, the anti-PD-1 antibody is pembrolizumab and is administered at a dosage between 100 and 300 mg every three weeks. In one embodiment, the pembrolizumab is administered at a dosage of 200 mg every three weeks.

In various embodiments, the LIV1-ADC is administered by intravenous infusion over a period of about 30 minutes. In various embodiments, the PD-1 antagonist, e.g., anti-PD-1 antibody is administered by intravenous infusion for a duration of approximately 30 minutes or approximately 60 minutes.

In various embodiments, the cancer comprises one or more cells that express PD-L1, PD-L2, or both PD-L1 and PD-L2. In various embodiments, the PD-L1 expression level of a tumor is at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 40%, at least about 50%, at least about 60%, at least about 70%, at least about 75%, at least about 80%, at least about 85%, at least about 90%, at least about 95%, or about 100%.

In various embodiments, at least about 0.01%, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the tumor cells express LIV-1

In various embodiments, the LIV-1 expression is measured by a FDA approved test.

In various embodiments, the LIV-1-ADC is ladiratuzumab vedotin and is administered at 2.5 mg/kg, and the anti-PD-1 antibody is pembrolizumab, and is administered at a dose of 1-2 mg/kg, or 100-300 mg once every three weeks. In one embodiment, the pembrolizumab is administered at a dose of 200 mg every three weeks. In another embodiment, the pembrolizumab is administered at a dose of 400 mg every six weeks.

In various embodiments, the LIV-1-ADC is ladiratuzumab vedotin and is administered at 2.0 mg/kg, and the anti-PD-1 antibody is pembrolizumab, and is administered at a dose of 1-2 mg/kg, or 100-300 mg once every three weeks. In one embodiment, the pembrolizumab is administered at a dose of 200 mg once every three weeks. In another embodiment, the pembrolizumab is administered at a dose of 400 mg every six weeks.

In various embodiments, the LIV-1-ADC is ladiratuzumab vedotin and is administered at 1.0 mg/kg, and the anti-PD-1 antibody is pembrolizumab and is administered at a dose of 1-2 mg/kg, or 100-300 mg once every three weeks. In one embodiment, the pembrolizumab is administered at a dose of 200 mg once every three weeks. In another embodiment, the pembrolizumab is administered at a dose of 400 mg every six weeks.

In various embodiments, the LIV-1-ADC is ladiratuzumab vedotin and is administered at 1.25 mg/kg, and the anti-PD-1 antibody is pembrolizumab and is administered at a dose of 1-2 mg/kg, or 100-300 mg once every three weeks. In one embodiment, the pembrolizumab is administered at a dose of 200 mg once every three weeks. In another embodiment, the pembrolizumab is administered at a dose of 400 mg every six weeks.

In various embodiments, the LIV-1 ADC is given every three weeks when administered in combination with a PD-1 antagonist, such as an anti-PD-1 antibody.

In various embodiments, the LIV-1 ADC is given once weekly when administered in combination with a PD-1 antagonist, such as an anti-PD-1 antibody.

In various embodiments, the LIV-1 ADC is administered on Days 1, 8, and 15 of a three week cycle, and pembrolizumab is administered Day 1 of each three week cycle. In various embodiments, the LIV-1 ADC is administered at 1.0 mg/kg or at 1.25 mg/kg on Days 1, 8, and 15 of a three week cycle, and pembrolizumab is administered at 200 mg on Day 1 of each three week cycle. In one embodiment, the LIV-1-ADC is administered at a dosage of 1.75 mg/kg on Days 1, 8, and 15 of a three week cycle, and optionally pembrolizumab is administered at 200 mg on Day 1 of each three week cycle.

Also provided is a method of treating a solid tumor that has metastasized or is at risk of metastasizing comprising administering to the subject a LIV-1 antibody drug conjugate (LIV-1-ADC), wherein the LIV-1-ADC is administered at a dosage between 1.0 mg/kg and 4 mg/kg of the subject's body weight. In various embodiments, the LIV-1-ADC is administered at a dosage of 1.0 mg/kg of the subject's body weight, or at a dosage of 1.25 mg/kg of the subject's body weight.

In various embodiments, the solid tumor is selected from the group consisting of non-small cell lung carcinoma (NSCLC) (squamous & non-squamous), small cell lung cancer, gastric/esophagogastric junction (GEJ) adenocarcinoma, esophageal squamous cell carcinoma, and head & neck squamous cell carcinoma.

Further contemplated herein is a method of treating a solid tumor selected from the group consisting of non-small cell lung carcinoma (NSCLC) (squamous & non-squamous), small cell lung cancer, gastric/esophagogastric junction (GES) adenocarcinoma, esophageal squamous cell carcinoma, and head & neck squamous cell carcinoma comprising administering to the subject a LIV-1 antibody drug conjugate (LIV-1-ADC).

In various embodiments, the LIV-1-ADC is administered at a dosage between 1.0 mg/kg and 4 mg/kg of the subject's body weight. In various embodiments, the LIV-1-ADC is administered at a dosage of 1.0 mg/kg of the subject's body weight, or at a dosage of 1.25 mg/kg of the subject's body weight.

In various embodiments, the LIV-1-ADC is administered at a dose of 2.0 to 2.5 mg/kg of the subject body weight.

In various embodiments, the LIV-1-ADC is administered once weekly or every three weeks.

In various embodiments, the subject is an adult patient.

In various embodiments, treatment with LIV-1-ADC and PD-1 antagonist increases the level of CD8+ T cell, dendritic cells, and/or macrophages in a tumor and/or tumor microenvironment. In various embodiments, treatment with LIV-1-ADC and PD-1 antagonist increases the level of macrophages in the tumor and/or tumor microenvironment. In various embodiments, treatment with LIV-1-ADC and PD-1 antagonist increases the level of CD8+ T cells in the tumor and/or tumor microenvironment. In various embodiments, treatment with LIV-1-ADC and PD-1 antagonist increases the level of dendritic cells in the tumor and/or tumor microenvironment. In various embodiments, the PD-1 antagonist is an anti-PD-1 antibody or an anti-PD-L1 antibody.

In various embodiments, treatment with LIV-1-ADC and PD-1 antagonist increases the expression of immune activation genes, e.g., in a tumor or more or more cells of a tumor. Immune activation genes include those associated with immune cells such as CD4+ T cells, CD8+ T cells, macrophages, and dendritic cells. In various embodiments, the immune activation gene is an MHC gene, a cytokine gene, a chemokine gene, a lectin gene, SIGLEC1, MS4A4A, CD163, CXCL12, IL-18, and/or APOE. In various embodiments, the immune activation genes are a HLA-DMA, HLA-DOA and IL-18.

It is specifically provided herein that all aspects of the disclosure described above with the methods of treatment are applicable to the anti-LIV-1 antibody drug conjugate combination therapy for use in any of the indications described above.

It is understood that each feature or embodiment, or combination, described herein is a non-limiting, illustrative example of any of the aspects of the disclosure and, as such, is meant to be combinable with any other feature or embodiment, or combination, described herein. For example, where features are described with language such as “one embodiment”, “some embodiments”, “certain embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination. Such features or combinations of features apply to any of the aspects of the disclosure. Where examples of values falling within ranges are disclosed, any of these examples are contemplated as possible endpoints of a range, any and all numeric values between such endpoints are contemplated, and any and all combinations of upper and lower endpoints are envisioned.

### BRIEF DESCRIPTION OF THE DRAWINGS

FIG. 1 shows the maximum change in tumor burden of treated patients as measured by the % change from baseline.

FIG. 2 shows the change in tumor burden over time (% change from baseline).

FIGS. 3A and 3B show efficacy of LIV1-ADC+pembrolizumab treatment in subjects who had previously received therapy (FIG. 3A, prior (neo) adjuvant therapy) compared to de novo (FIG. 3B) patients who had not been treated previously.

FIG. 4 is a volcano plot showing differentially expressed genes in tumor tissue induced by LV monotherapy.

FIGS. 5A-5C show increased macrophage infiltration and PD-L1 expression and induction of MHC and co-stimulatory molecules by LV monotherapy. FIG. 5A: Summary graphs showing CD68 IHC results (% of cells positive for CD68 in tumor stroma), p=9.1e-07.

FIG. 5B: PD-L1 CPS scores in all LVA-001 patients with evaluable samples (N=79 for CD68; N=73 for PD-L1), p=0.0068. FIG. 5C: Summary graphs showing induction of MHCII genes (HLA-DRB1 (FDR=3.76e-5) and HLA-DQA1(FDR=0.005)) and co-stimulatory molecules (CD80 (FDR=0.002) and CD86 (FDR=1.69e-6)) in tumor tissues by RNAseq analysis.

FIG. 6 is a volcano plot showing differentially expressed genes in tumor tissue induced by LV plus pembro.

FIG. 7 shows a comparison of xCell analysis (RNAseq) results on tumor infiltrating immune cells (macrophage, DC, and CD8 T cells) and CD274 (PD-L1) gene expression in LVA-001 (LV only) and LVA-002 (LV+pembro). P-values for xCell gene signatures were calculated using a paired t-test. For PD-L1, the FDR is derived from the differential expression analysis comparing C1D5/C1D15 and baseline. N=59 for LVA-001; N=16 for LVA-002.

FIGS. 8A-8B show a comparison of IHC analysis on tumor infiltrating immune cells (CD3+ T cells and CD68+ macrophages) (FIG. 8A) and CD8+ T cells and CD274 (PD-L1 CPS) (FIG. 8B) staining in LVA-001 and LVA-002. P-values were calculated by a Wilcoxon signed-rank sum using the “coin” permutation framework in R9. On-treatment biopsies were collected around C1D5 in LVA-001 and around C1D5 or C1D15 in LVA-002.

FIG. 9 depicts a xCell gene signature analysis of RNAseq data showed that induction of genes associated with CD4 T cells (especially Effector Memory CD4 T cells) and conventional DC (cDC) are associated with clinical responses. P-values were calculated by using a likelihood ratio test of two nested linear models.

FIG. 10 depicts that induction of MHCI genes (HLA-DMA and HLA-DOA) and IL-18 are associated with clinical responses. FDRs are calculated by a Benjamini-Hochberg correction of multiple testing for p-values generated by a DESeq-based likelihood ratio test of two nested models.

### DETAILED DESCRIPTION

The present disclosure provides methods for treating a cancer with an anti-LIV1 antibody drug conjugate (LIV1-ADC) in combination with a PD-1 antagonist. The present disclose shows for the first time that the LIV1-ADC+PD-1 antagonist combination therapy is safe and effective at treating cancer.

Unless otherwise defined, all technical and scientific terms used herein have the same meaning as commonly understood by one of ordinary skill in the art to which this disclosure belongs. The following references provide one of skill with a general definition of many of the terms used in this disclosure: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger et al. (eds.), Springer Verlag (1991); and Hale & Marham, THE HARPER COLLINS DICTIONARY OF BIOLOGY (1991).

Each publication, patent application, patent, and other reference cited herein is incorporated by reference in its entirety to the extent that it is not inconsistent with the present disclosure.

As used herein and in the appended claims, the singular forms “a,” “and,” and “the” include plural referents unless the context clearly dictates otherwise. Thus, for example, reference to “a derivative” includes a plurality of such derivatives and reference to “a subject” includes reference to one or more subjects and so forth.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

“Cancer” as used herein refers to a disease or disorder in which cells of a certain type, for example breast cells, exhibit abnormal cell growth, resulting in tumors, or large aggregations of abnormally dividing cells. Cancer encompasses one or more tumors, the tumor microenvironment which surrounds the tumor, as well as cells of the cancer or tumor that break off from an initial tumor in one site and migrate, or metastasize, to a different part of the body.

“Treatment” refers to prophylactic treatment or therapeutic treatment or diagnostic treatment. In certain embodiments, “treatment” refers to administration of a compound or composition to a subject for therapeutic, prophylactic or diagnostic purposes.

A “prophylactic” treatment is a treatment administered to a subject who does not exhibit signs of a disease or exhibits only early signs of the disease, for the purpose of decreasing the risk of developing pathology. The compounds or compositions of the disclosure may be given as a prophylactic treatment to reduce the likelihood of developing a pathology or to minimize the severity of the pathology, if developed.

A “therapeutic” treatment is a treatment administered to a subject who exhibits signs or symptoms of pathology for the purpose of diminishing or eliminating those signs or symptoms. The signs or symptoms may be biochemical, cellular, histological, functional or physical, subjective or objective.

The term “effective amount” or “therapeutically effective amount” refers to the amount of a LIV-1-ADC, e.g., SGN-LIV1A, that is sufficient to inhibit the occurrence or ameliorate one or more clinical or diagnostic symptoms of a LIV-1-associated disorder in a subject. An effective amount of an agent is administered according to the methods described herein in an “effective regimen.” The term “effective regimen” refers to a combination of amount of the agent and dosage frequency adequate to maintain high LIV-1 occupancy, which may accomplish treatment or prevention of a LIV-1-associated disorder. In a preferred embodiment, an effective regimen maintains near complete, e.g., greater than 90%, LIV-1 occupancy on LIV-1-expressing cells during dosing intervals. Effective amount also refers to the amount of PD-1 antagonist, e.g., anti-PD-1 or anti-PDL1 antibody, that is sufficient to inhibit the occurrence or ameliorate one or more clinical or diagnostic symptoms of a PD-1/PD-L1-associated disorder in a subject

“Cytotoxic effect” refers to the depletion, elimination and/or the killing of a target cell. A “cytotoxic agent” refers to an agent that has a cytotoxic effect on a cell. Cytotoxic agents can be conjugated to an antibody or administered in combination with an antibody. “Cytotoxic therapy” refers to treatment with a cytotoxic agent.

“Cytostatic effect” refers to the inhibition of cell proliferation. A “cytostatic agent” refers to an agent that has a cytostatic effect on a cell, thereby inhibiting the growth and/or expansion of a specific subset of cells. Cytostatic agents can be conjugated to an antibody or administered in combination with an antibody.

“PD-1 antagonist” as used herein refer to any chemical compound or biological molecule that blocks the binding of PD-L1 expressed on a cancer cell to PD-1 expressed on an immune cell (T cell, Bc cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for Pd-1; PDCD1L1, PDL1, B7H1, B7-4, CD274 and B7-H for PD-L1; and PDCD1L2, PDL2, B7-Dc, Btdc and CD273 for PD-L2. In any of the treatments methods and disclosed uses in which a human individual is being treated, the PD-1 antagonist blocks binding of human PD-L1 to human PD-1, and preferably blocks binding of both human PD-L1 and PD-L2 to human PD-1. Human PD-1 amino add sequences can be found in NCBI Locus No.: NP 005009. Human PD-L1 and PD-L2 amino acid sequences can be found in NCBI Locus No.: NP 054862 and NP 07955, respectively.

The term “checkpoint inhibitor” as used herein refers to a molecule or therapeutic that blocks certain proteins made by some types of immune system cells, such as T cells, and some cancer cells. These proteins help keep immune responses in check and can keep T cells from killing cancer cells. Examples of checkpoint proteins found on T cells or cancer cells include PD-1, PD-L1, PD-L2, CD28, CTLA-4, B7-1, B7-2 (see National Cancer Institute Dictionary of Cancer Terms) as well as ICOS, BTLA, TIM3 and LAG3.

“Programmed Death-1” (PD-1) refers to an immunoinhibitory receptor belonging to the CD28 family. PD-1 is expressed on previously activated T cells in vivo, and binds to two ligands, PD-L1 and PD-L2. The complete human PD-1 sequence can be found under GenBank Accession No. U64863.

“Programmed Death Ligand-1” (PD-L1) and PD-L2 are cell surface ligands for PD-1 that downregulate T cell activation and cytokine secretion upon binding to PD-1. The complete human PD-L1 sequence can be found under GenBank Accession No. Q9NZQ7.

The terms “specific binding” and “specifically binds” mean that an antibody as described herein, e.g., anti-LIV-1 antibody or anti-PD-1 antibody, will react, in a highly selective manner, with its corresponding target and not with the multitude of other antigens.

The term “monoclonal antibody” refers to an antibody that is derived from a single cell clone, including any eukaryotic or prokaryotic cell clone, or a phage clone, and not the method by which it is produced. Thus, the term “monoclonal antibody” as used herein is not limited to antibodies produced through hybridoma technology.

The term “pharmaceutically acceptable” as used herein refers to those compounds, materials, compositions, and/or dosage forms that are, within the scope of sound medical judgment, suitable for contact with the tissues of human beings and animals without excessive toxicity, irritation, allergic response, or other problems or complications commensurate with a reasonable benefit/risk ratio. The term “pharmaceutically compatible ingredient” refers to a pharmaceutically acceptable diluent, adjuvant, excipient, or vehicle with which an anti-LIV-1 antibody is administered.

The phrase “pharmaceutically acceptable salt,” refers to pharmaceutically acceptable organic or inorganic salts of an antibody described herein, e.g., anti-LIV-1 antibody or conjugate thereof or agent administered with an anti-LIV-1 antibody or LIV1-ADC. Exemplary salts include sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p toluenesulfonate, and pamoate (i.e., 1,1′ methylene bis-(2 hydroxy 3 naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Multiple counter ions may occur in instances where multiple charged atoms are part of the pharmaceutically acceptable salt. Hence, a pharmaceutically acceptable salt can have one or more charged atoms and/or one or more counterion. The term “pharmaceutically compatible ingredient” refers to a pharmaceutically acceptable diluent, adjuvant, excipient, or vehicle with which an antibody-drug conjugate is administered.

Except when noted, the terms “subject” or “patient” are used interchangeably and refer to mammals such as human patients and non-human primates, as well as experimental animals such as rabbits, dogs, cats, rats, mice, and other animals. Accordingly, the term “subject” or “patient” as used herein means any mammalian patient or subject to which the LIV1-ADC of the disclosure can be administered. In preferred embodiments, the terms subject or patient are used to refer to human patients. Subjects of the present invention include those that have been diagnosed with a LIV-1 expressing cancer, including, for example, breast cancer, prostate cancer, ovarian cancer, endrometrial cancer, pancreatic cancer, lung cancer, cervical cancer, a melanoma, or squamous cell carcinoma. In certain embodiments, the subject will have a refractory or relapsed LIV-1 expressing cancer or a metastatic LIV-1 expressing cancer.

Compositions or methods “comprising” one or more recited elements may include other elements not specifically recited. For example, a composition that comprises antibody may contain the antibody alone or in combination with other ingredients.

The terms “identical” or “percent identity,” in the context of two or more nucleic acids or polypeptide sequences, refer to two or more sequences or subsequences that are the same or have a specified percentage of nucleotides or amino acid residues that are the same, when compared and aligned for maximum correspondence. To determine the percent identity, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In certain embodiments, the two sequences are the same length.

The term “substantially identical,” in the context of two nucleic acids or polypeptides, refers to two or more sequences or subsequences that have at least 70% or at least 75% identity; more typically at least 80% or at least 85% identity; and even more typically at least 90%, at least 95%, or at least 98% identity (for example, as determined using one of the methods set forth below).

The determination of percent identity between two sequences can be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul, et al., 1990, J. Mol. Biol. 215:403-410. BLAST nucleotide searches can be performed with the NBLAST program, score=100, wordlength=12 to obtain nucleotide sequences homologous to a nucleic acid encoding a protein of interest. BLAST protein searches can be performed with the XBLAST program, score=50, wordlength=3 to obtain amino acid sequences homologous to protein of interest. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Id.). Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. Additional algorithms for sequence analysis are known in the art and include ADVANCE and ADAM as described in Torellis and Robotti, 1994, Comput. Appl. Biosci. 10:3-5; and FASTA described in Pearson and Lipman, 1988, Proc. Natl. Acad. Sci. 85:2444-8. Alternatively, protein sequence alignment may be carried out using the CLUSTAL W algorithm, as described by Higgins et al., 1996, Methods Enzymol. 266:383-402.

The abbreviation “MMAE” refers to monomethyl auristatin E.

The abbreviations “vc” and “val-cit” refer to the dipeptide valine-citrulline.

The abbreviation “PAB” refers to a self-immolative spacer:

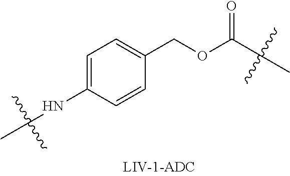 [SMILES: *Nc1ccc(COC(*)=O)cc1]

Antibodies of the present disclosure may be described or specified in terms of the particular CDRs or variable regions they comprise. Additionally, antibodies of the present disclosure may also be described or specified in terms of their primary structures. Antibodies having at least 50%, at least 55%, at least 60%, at least 65%, at least 70%, at least 75%, at least 80%, at least 85%, at least 90%, at least 95% and most preferably at least 98% identity (as calculated using methods known in the art and described herein) to the variable regions described herein are also included in the present disclosure. Antibodies useful in the present methods disclosure may also be described or specified in terms of their binding affinity. Preferred binding affinities include those with a dissociation constant or Kd less than 5×10 2 M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸M, 5×10⁻⁹ M, 10⁻¹⁰ M, 5×10⁻¹⁰ M, 10⁻¹¹ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

The antibodies also include derivatives that are modified, i.e., by the covalent attachment of any type of molecule to the antibody such that covalent attachment does not prevent the antibody from binding to LIV-1 or from exerting a cytostatic or cytotoxic effect on tumor cells. For example, but not by way of limitation, the antibody derivatives include antibodies that have been modified, e.g., by glycosylation, acetylation, PEGylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The antibodies described herein may be generated by any suitable method known in the art.

As used herein, a LIV-1-antibody drug conjugate (LIV-1-ADC; LV) includes an antibody specific for the human LIV-1 protein conjugated to a cytotoxic agent. An exemplary human LIV-1 sequence is assigned Swiss Prot accession number Q13433. Q13433 is included herein as SEQ ID NO: 1. Three variant isoforms and one polymorphism are known. A second version of the human LIV-1 protein, accession number AAA96258.2, is included herein as SEQ ID NO:2. Four extracellular domains are bounded by residues 29-325, 377-423, 679-686 and 746-755 of Q13433 respectively.

SGN-LIV1A is a LIV-1-directed antibody-drug conjugate (ADC) produced by the conjugation of the drug linker vcMMAE (monomethyl auristatin E with a valine-citrulline linker) to the humanized antibody hLIV22. hLIV22 is a humanized form of the mouse BR2-22a anti-body, described in U.S. Pat. No. 9,228,026. The hLIV22 antibody is essentially the same as BR2-22a within experimental error and contains seven back mutations. Methods of making the hLIV22 antibody are also disclosed in U.S. Pat. No. 9,228,026. The amino acid sequence of the light chain variable region of hLIV22 is provided herein as SEQ ID NO: 3. The amino acid sequence of the heavy chain variable region of hLIV22 is provided herein as SEQ ID NO: 4.

Antibodies having at least 50%, at least 55%, at least 60%, at least 65%, at least 70%, at least 75%, at least 80%, at least 85%, at least 90%, at least 95% and most preferably at least 98% identity (as calculated using methods known in the art and described herein) to the variable regions of hLIV22 are also included in the present disclosure, and preferably include the CDRs of hLIV22.

In various embodiments, the anti-LIV-1 antibody of the LIV-1-ADC comprises i) a heavy chain CDR1, CDR2, and CDR3 of the hLIV22 antibody and ii) a light chain CDR1, CDR2, and CDR3 of the hLIV22 antibody.

In various embodiments, the anti-LIV-1 antibody of the LIV1-ADC comprises i) an amino acid sequence at least 85% identical to a heavy chain variable region set out in SEQ ID NO: 4 and ii) an amino acid sequence at least 85% identical to a light chain variable region set out in SEQ ID NO: 3. In various embodiments, the anti-LIV-1 antibody of LIV-1-ADC comprises i) an amino acid sequence at least 90% identical to a heavy chain variable region set out in SEQ ID NO: 4 and ii) an amino acid sequence at least 90% identical to a light chain variable region set out in SEQ ID NO: 3. In various embodiments, the anti-LIV-1 antibody of LIV-1-ADC comprises i) an amino acid sequence at least 95% identical to a heavy chain variable region set out in SEQ ID NO: 4 and ii) an amino acid sequence at least 95% identical to a light chain variable region set out in SEQ ID NO: 3. In various embodiments, the anti-LIV-1 antibody of LIV-1-ADC comprises i) a heavy chain variable region amino acid sequence set out in SEQ ID NO: 4 and ii) a light chain variable region amino acid sequence set out in SEQ ID NO: 3.

In various embodiments, the anti-LIV-1 antibody (i) cross-competes with an antibody comprising a heavy chain variable region set out in SEQ ID NO: 4; and ii) a light chain variable region set out in SEQ ID NO: 3, for binding to LIV-1; or (ii) binds to the same epitope as an antibody comprising a heavy chain variable region set out in SEQ ID NO: 4; and ii) a light chain variable region set out in SEQ ID NO: 3, for binding to LIV-1.

Synthesis and conjugation of the drug linker vcMMAE (shown below; also referred to as 1006) are further described in U.S. Pat. No. 9,228,026 and US Patent Pub. No. 20050238649.

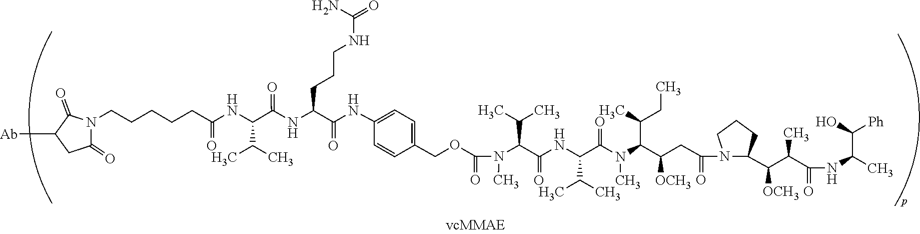

Ladiratuzumab vedotin is a LIV-1-directed antibody-drug conjugate consisting of three components: (i) the humanized antibody hLIV22, specific for human LIV-1, (ii) the microtubule disrupting agent MMAE, and (iii) a protease-cleavable linker that covalently attaches MMAE to hLIV22. The drug to antibody ratio or drug loading is represented by “p” in the structure of ladiratuzumab vedotin.

In various embodiments, the LIV-1 antibody drug conjugate is ladiratuzumab vedotin.

### Combination Therapy of Chemotherapeutic Agents and LIV-1-ADC

Cancer can be treated using a combination of LIV1-ADC and a PD-1 antagonist. Examples of PD-1 antagonists include antibodies such as anti-PD-1 antibodies (e.g., MED10680, AMP-224, nivolumab, pembrolizumab, and pidilizumab) and anti-PD-L1 antibodies (e.g., MED14736 and MPDL3280A). WO 2017161007/describes use of a combination of LIV-ADC with chemotherapeutics to treat cancer.

It is contemplated that combination therapy with LIV1-ADC can also be carried out with other checkpoint inhibitors. Examples of additional checkpoint inhibitors (inhibitors that block immune checkpoints) that may be used include anti-CTLA4 antibodies (e.g., ipilimumab and tremelimumab), B7-DC-Fc, LAG3 and TIM3. WO 2017/161007 describes use of a combination of LIV-ADC with chemotherapeutics to treat cancer.

Human monoclonal antibodies that bind to PD-1 have been disclosed in U.S. Pat. Nos. 8,008,449, 6,808,710, 7,488,802, 8,168,757 and 8,354,509, and PCT Publication No. WO 2012/145493.

In one embodiment, the anti-PD-1 antibody is pembrolizumab. Pembrolizumab (“KEYTRUDA®”, lambrolizumab, MK-3475) is a humanized monoclonal IgG4 antibody directed against human cell surface receptor PD-1 (programmed death-1 or programmed cell death-1). Pembrolizumab is described, for example, in PCT International Application Publication No. WO 2008/156712 and has the structure as set forth in WHO Drug Information, Vol. 27, No. 2, pages 161-162 (2013). Pembrolizumab has been approved by the FDA for the treatment of relapsed or refractory melanoma and advanced NSCLC. In various embodiments, the anti-PD-1 antibody or antigen-binding portion thereof cross-competes with pembrolizumab. In various embodiments, the anti-PD-1 antibody binds to the same epitope as pembrolizumab. In various embodiments, the anti-PD-1 antibody has the same CDRs as pembrolizumab.

A “pembrolizumab variant” as used herein refers to a monoclonal antibody that comprises heavy chain and light chain sequences that are substantially identical to those in pembrolizumab, except for having three, two or one conservative amino acid substitutions at positions that are located outside of the light chain CDRs and six, five, four, three, two or one conservative amino acid substitutions that are located outside of the heavy chain CDRs, e.g., the variant positions are located in the framework (FR) regions or the constant region, and optionally has a deletion of the C-terminal lysine residue of the heavy chain. In other words, pembrolizumab and a pembrolizumab variant comprise identical CDR sequences, but differ from each other due to having a conservative amino acid substitution at no more than three or six other positions in their full length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

Table 1 provides a list of the amino acid sequences of exemplary anti-PD1 antibodies for use in the methods disclosed herein.

TABLE 1
Exemplary anti-human PD-1 antibodies
A. Comprises light and heavy chain CDRs of hPD-1.09A in WO2008/156712 (light
and heavy chain CDRs of pembrolizumab)

| | |
|---|---|
| CDRL1 | RASKGVSTSGYSYLH |
| | SEQ ID NO: 5 |
| CDRL2 | LASYLES |
| | SEQ ID NO: 6 |
| CDRL3 | QHSRDLPLT |
| | SEQ ID NO: 7 |
| CDRH1 | NYYMY |
| | SEQ ID NO: 8 |
| CDRH2 | GINPSNGGTNFNEKFKN |
| | SEQ ID NO: 9 |
| CDRH3 | RDYRFDMGFDY |
| | SEQ ID NO: 10 |

B. Comprises light and heavy chain CDRs of hPD-1.08A in WO2008/156712

| | |
|---|---|
| CDRL1 | RASKSVSTSGFSYLH |
| | SEQ ID NO: 11 |
| CDRL2 | LASNLES |
| | SEQ ID NO: 12 |
| CDRL3 | QHSWELPLT |
| | SEQ ID NO: 13 |
| CDRH1 | SYYLY |
| | SEQ ID NO: 14 |
| CDRH2 | GVNPSNGGTNFSEKFKS |
| | SEQ ID NO: 15 |
| CDRH3 | RDSNYDGGFDY |
| | SEQ ID NO: 16 |

C. Comprises the mature h109A heavy chain variable region (V_{H}) and one of
the mature K09A light chain variable (V_{L}) regions in WO 2008/156712

| | |
|---|---|
| Heavy chain V_{H} | QVQLVQSGVEVKKPGASVKVSCKASGYTFTNYYMYWVRQAPGQ |
| | GLEWMGGINPSNGGTNFNEKFKNRVTLTTDSSTTTAYMELKSLQ |
| | FDDTAVYYCARRDYRFDMGFDYWGQGTTVTVSS |
| | SEQ ID NO: 17 (VH of pembrolizumab) |
| Light Chain V_{L} | EIVLTQSPATLSLSPGERATLSCRASKGVSTSGYSYLHWYQQKP |
| | GQAPRLLIYLASYLESGVPARFSGSGSGTDFTLTISSLEPEDFAVY |
| | YCQHSRDLPLTFGGGTKVEIK |
| | SEQ ID NO: 18 (VL of pembrolizumab) or |
| | EIVLTQSPLSLPVTPGEPASISCRASKGVSTSGYSYLHWYLQKPG |
| | QSPQLLIYLASYLESGVPDRFSGSGSGTDFTLKISRVEAEDVGVY |
| | YCQHSRDLPLTFGQGTKLEIK (SEQ ID NO: 19) or |
| | DIVMTQTPLSLPVTPGEPASISCRASKGVSTSGYSYLHWYLQKPG |
| | QSPQLLIYLASYLESGVPDRFSGSGSGTAFTLKISRVEAEDVGLYY |
| | CQHSRDLPLTFGQGTKLEIK |
| | SEQ ID NO: 20 |

D. Comprises the mature 409 heavy chain and one of the mature K09A light
chains in WO 2008/156712

| | |
|---|---|
| Heavy chain | QVQLVQSGVEVKKPGASVKVSCKASGYTFTNYYMYWVRQAPGQ |
| | GLEWMGGINPSNGGTNFNEKFKNRVTLTTDSSTTTAYMELKSLQ |
| | FDDTAVYYCARRDYRFDMGFDYWGQGTTVTVSSASTKGPSVFP |
| | LAPCSRSTSESTAALGCLVKDYFPEPVTVSWNSGALTSGVHTFP |
| | AVLQSSGLYSLSSWTVPSSSLGTKTYTCNVDHKPSNTKVDKRVE |
| | SKYGPPCPPCPAPEFLGGPSVFLFPPKPKDTLMISRTPEVTCVVV |
| | DVSQEDPEVQFNWYVDGVEVHNAKTKPREEQFNSTYRWSVLT |
| | VLHQDWLNGKEYKCKVSNKGLPSSIEKTISKAKGQPREPQVYTLP |
| | PSQEEMTKNQVSLTCLVKGFYPSDIAVEWESNGQPENNYKTTPP |
| | VLDSDGSFFLYSRLTVDKSRWQEGNVFSCSVMHEALHNHYTQK |
| | SLSLSLGK SEQ ID NO: 21 (heavy chain of pembrolizumab) |
| Light chain | EIVLTQSPATLSLSPGERATLSCRASKGVSTSGYSYLHWYQQKP |
| | GQAPRLLIYLASYLESGVPARFSGSGSGTDFTLTISSLEPEDFAVY |
| | YCQHSRDLPLTFGGGTKVEIKRTVAAPSVFIFPPSDEQLKSGTAS |
| | VVCLLNNFYPREAKVQWKVDNALQSGNSQESVTEQDSKDSTYS |
| | LSSTLTLSKADYEKHKVYACEVTHQGLSSPVTKSFNRGEC |
| | SEQ ID NO: 22 (light chain of pembrolizumab) or |
| | EIVLTQSPLSLPVTPGEPASISCRASKGVSTSGYSYLHWYLQKPG |
| | QSPQLLIYLASYLESGVPDRFSGSGSGTDFTLKISRVEAEDVGVY |
| | YCQHSRDLPLTFGQGTKLEIKRTVAAPSVFIFPPSDEQLKSGTAS |
| | VVCLLNNFYPREAKVQWKVDNALQSGNSQESVTEQDSKDSTYS |
| | LSSTLTLSKADYEKHKVYACEVTHQGLSSPVTKSFNRGEC |
| | SEQ ID NO: 23 or |
| | DIVMTQTPLSLPVTPGEPASISCRASKGVSTSGYSYLHWYLQKPG |
| | QSPQLLIYLASYLESGVPDRFSGSGSGTAFTLKISRVEAEDVGLYY |
| | CQHSRDLPLTFGQGTKLEIKRTVAAPSVFIFPPSDEQLKSGTASV |
| | VCLLNNFYPREAKVQWKVDNALQSGNSQESVTEQDSKDSTYSL |
| | SSTLTLSKADYEKHKVYACEVTHQGLSSPVTKSFNRGEC |
| | SEQ ID NO: 24 |

Also contemplated are variants of the above anti-PD-1 antibody heavy and light chain regions set out in Table 1, wherein the variants retain the sequence of the CDRs within the parental variable region, e.g., as disclosed in Table 1. In various embodiments, the anti-PD-1 antibody comprises i) an amino acid sequence at least 85%, 90%, or 95% identical to a heavy chain variable region set out in Table 1 and ii) an amino acid sequence at least 85%, 90% or 95% identical to a light chain variable region set out in Table 1.

In one embodiment, the anti-PD-1 antibody is nivolumab. Nivolumab (also known as “OPDIVO®”; formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), blocking the down-regulation of antitumor T-cell functions (U.S. Pat. No. 8,008,449; Wang et al., 2014 Cancer Immunol Res. 2(9):846-56). Nivolumab has the structure described in WHO Drug Information, Vol. 27, No. 1, pages 68-69 (2013), herein incorporated by reference. In another embodiment, the anti-PD-1 antibody or fragment thereof cross-competes with nivolumab. In some embodiments, the anti-PD-1 antibody binds to the same epitope as nivolumab. In certain embodiments, the anti-PD-1 antibody has the same CDRs as nivolumab.

Additional anti-PD-1 antibodies contemplated for use herein include MED10680 (U.S. Pat. No. 8,609,089), BGB-A317 (U.S. Patent Publ. No. 2015/0079109), INCSHR1210 (SHR-1210) (WO2015/085847), REGN-2810 (WO2015/112800) PDR001 (WO2015/112900), TSR-042 (ANB011) (WO2014/179664), and STI-1110 (WO2014/194302).

In various embodiments, the anti-PD-1 antibody or antigen-binding portion thereof is a chimeric, humanized or human monoclonal antibody or a portion thereof. In various embodiments, the antibody is a human or humanized antibody. Antibodies having an IgG1, IgG2, IgG3, or IgG4 isotype are contemplated.

In various embodiments, the anti-PD-1 antibody (i) cross-competes with nivolumab or pembrolizumab for binding to human PD-1; (ii) binds to the same epitope as nivolumab or pembrolizumab; (iii) is nivolumab; or (iv) is pembrolizumab.

In various embodiments, the anti-PD-1 antibody (i) cross-competes with nivolumab or pembrolizumab for binding to human PD-1; (ii) binds to the same epitope as nivolumab or pembrolizumab; (iii) is nivolumab; (iv) is pembrolizumab; or (v) is a pembrolizumab variant.

Treatment with the LIV1-ADC+PD-1 antagonist combination therapies of the invention can be further combined with additional chemotherapy, radiation, stem cell treatment, surgery other treatments effective against the disorder being treated. Useful classes of other agents that can be administered with the combination therapies of the invention include, for example, antibodies to other receptors expressed on cancerous cells, including antibodies to the HER2 receptor (e.g., Trastuzumab, rastuzumab emtansine (KADCYLA®, Genentech, South San Francisco, Calif.), antitubulin agents (e.g., auristatins), pertuzumab (PERJETA®, Genentech, South San Francisco, Calif.)), or other antibody drug conjugates such as sacituzumab govitecan, DNA minor groove binders, DNA replication inhibitors, alkylating agents (e.g., platinum complexes such as cis-platin, mono(platinum), bis(platinum) and tri-nuclear platinum complexes and carboplatin), anthracyclines, antibiotics, antifolates, antimetabolites, chemotherapy sensitizers, duocarmycins, etoposides, fluorinated pyrimidines, ionophores, lexitropsins, nitrosoureas, platinols, pre-forming compounds, purine antimetabolites, puromycins, radiation sensitizers, steroids, taxanes, topoisomerase inhibitors, vinca alkaloids, and the like

In various embodiments, the treatment with LIV-1-ADC+PD-1 antagonist further comprises an additional chemotherapeutic agent, including, but not limited to, carboplatin, doxorubicin or paclitaxel, trastuzumab, an mTOR inhibitor (such as Everolimus). Carboplatin (PARAPLATIN®; Bristol Myers Squibb, New York, N.Y.) is an alkylating agent. Doxorubicin (ADRIAMYCIN®, RUBEX®, DOXIL®, MYOCEL®, or CAELYX®) is an anthracycline antibiotic with antineoplastic activity. Paclitaxel (ABRAXANE®; Celgene, Summit, N.J.) is a taxane that inhibits microtubule breakdown.

The combination of LIV-1-ADC and a PD-1 antagonist, e.g., anti-PD-1 antibody, can be given to subjects at levels that inhibit cancer cell growth, while at the same time are tolerated by the subject. In some embodiments, the combination of LIV1-ADC and a checkpoint inhibitor is synergistic or additive. For some combinations, each agent in the combination can be effectively administered at lower levels than when administered alone.

### Methods of Use

As discussed above, the combination therapies of the disclosure can be used to treat cancer. Some such cancers show detectable levels of LIV-1 measured at either the protein (e.g., by immunoassay using one of the exemplified antibodies) or mRNA level. Some such cancers show elevated levels of LIV-1 relative to noncancerous tissue of the same type, preferably from the same patient. An exemplary level of LIV-1 on cancer cells amenable to treatment is 5000-150,000 LIV-1 molecules per cell, although higher or lower levels can be treated. Optionally, a level of LIV-1 in a cancer is measured before performing treatment.

In various embodiments, the subject has a tumor comprising one or more cells that express LIV-1. In various embodiments, at least about 0.01%, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the tumor cells express LIV-1.

Exemplary dosages for LIV-1-ADC are 0.1 mg/kg to 50 mg/kg of the patient's body weight, more typically 1 mg/kg to 30 mg/kg, 1 mg/kg to 20 mg/kg, 1 mg/kg to 15 mg/kg, 1 mg/kg to 12 mg/kg, 1 mg/kg to 10 mg/kg, 2 mg/kg to 30 mg/kg, 2 mg/kg to 20 mg/kg, 2 mg/kg to 15 mg/kg, 2 mg/kg to 12 mg/kg, 2 mg/kg to 10 mg/kg, 3 mg/kg to 30 mg/kg, 3 mg/kg to 20 mg/kg, 3 mg/kg to 15 mg/kg, 3 mg/kg to 12 mg/kg, or 3 mg/kg to 10 mg/kg. In some methods, the patient is administered a dose of at least about 1.0 mg/kg, 1.25 mg/kg, 1.5 mg/kg, 1.75 mg/kg, 2 mg/kg, 2.5 mg/kg or 3 mg/kg, administered once weekly, or once every three weeks or greater. In one embodiment, the LIV-1-ADC is administered at a dosage of 1.0 mg/kg. In one embodiment, the LIV-1-ADC is administered at a dosage of 1.25 mg/kg. In one embodiment, the LIV-1-ADC is administered at a dosage of 1.5 mg/kg. In one embodiment, the LIV-1-ADC is administered at a dosage of 1.75 mg/kg. In one embodiment, the LIV-1-ADC is administered at a dosage of 2.0 mg/kg. In one embodiment, the LIV-1-ADC is administered at a dose of 2.5 mg/kg. In various embodiments, the LIV-1-ADC is administered at a dose of 1.0 mg/kg or 1.25 mg/kg weekly. In a further embodiment, LIV-1-ADC is administered at a dose of 0.5 mg/kg to 2.8 mg/kg, administered weekly or every three weeks. In a further embodiment, LIV-1-ADC is administered at a dose of 1.0 mg/kg to 1.75 mg/kg, administered weekly or every three weeks. In a further embodiment, LIV-1-ADC is administered at a dose of 2.0 mg/kg or 2.5 mg/kg, administered once every three weeks. The dosage depends on the frequency of administration, condition of the patient and response to prior treatment, if any, whether the treatment is prophylactic or therapeutic and whether the disorder is acute or chronic, among other factors.

It is also contemplated that the subject has a tumor expressing PD-1 or a ligand for PD-1, e.g., PD-L1 or PD-L2. Methods of measuring levels of PD-1, PD-L1 or PD-L2 known in the art are contemplated herein for determining levels of the molecules in a tumor cell. See, e.g., WO2017/210473.

In various embodiments, the PD-L1 expression level of a tumor is at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 40%, at least about 50%, at least about 60%, at least about 70%, at least about 75%, at least about 80%, at least about 85%, at least about 90%, at least about 95%, or about 100%.

In various embodiments, the anti-PD-1 antibody dose may be administered from at least about 0.1 mg/kg to at least about 10 mg/kg, from about 0.01 mg/kg to about 5 mg/kg, from about 1 mg/kg to about 5 mg/kg, from about 2 mg/kg to about 5 mg/kg, from about 1 mg/kg to about 3 mg/kg, or from about 7.5 mg/kg to about 12.5 mg/kg. In various embodiments, the anti-PD-1 antibody is given on a dose amount basis. In various embodiments, the dose of the anti-PD-1 antibody is from about 100-600 mg, from about 400-500 mg, from about 100-200 mg, from about 200-400 mg, or from about 100-300 mg. In various embodiments, the anti-PD-1 antibody is administered at a dose of about 60 mg, about 80 mg, about 100 mg, about 120 mg, about 130 mg, about 140 mg, about 160 mg, about 180 mg, about 200 mg, about 220 mg, about 240 mg, about 260 mg, about 280 mg, at least about 300 mg, about 320 mg, about 360 mg, about 400 mg, about 440 mg, about 480 mg, about 500 mg, about 550 mg, or about 600 mg.

In various embodiments, anti-LIV-1-ADC+PD-1 antagonist therapy further comprises administering another chemotherapeutic. In various embodiments, the additional chemotherapeutic is carboplatin, doxorubicin or paclitaxel. In combination with a PD-1 antagonist, and optionally, with carboplatin, doxirubicin, or paclitaxel LIV1-ADC is administered at a dose between 0.5 mg/kg and 6 mg/kg. Other appropriate dose ranges of LIV1-ADC in the combination are 1 mg/kg to 5 mg/kg, and 2 mg/kg to 3 mg/kg. In an embodiment, LIV1-ADC is administered at a dose of 1.0 mg/kg, 1.25 mg/kg, 1.5 mg/kg, 1.75 mg/kg, 2.0 mg/kg or 2.5 mg/kg in combination with a chemotherapeutic, such as carboplatin, doxirubicin, or paclitaxel. In various embodiments, LIV-1-ADC is administered at a dose of 0.5 mg/kg to 2.8 mg/kg, or a dose of 1.0 mg/kg to 1.75 mg/kg, in combination with a chemotherapeutic, such as carboplatin, doxirubicin, or paclitaxel. In combination with LIV1-ADC and a PD-1 antagonist, carboplatin is administered at a dose between 100 mg/m² and 950 mg/m². Other appropriate dose ranges of carboplatin in the combination are 200 mg/m² to 750 mg/m², and 300 mg/m² to 600 mg/m². In an embodiment, carboplatin is administered at a dose of 300 mg/m² in combination with LIV1-ADC and a PD-1 antagonist. In another embodiment, carboplatin is administered at a dose of AUC 6 IV in combination with LIV-1-ADC and PD-1 antagonist.

In combination with LIV-1-ADC and a PD-1 antagonist, doxorubicin is administered at a dose between 30 mg/m² and 90 mg/m². Other appropriate dose ranges of doxorubicin in the combination are 40 mg/m² to 80 mg/m², and 60 mg/m² to 75 mg/m². In an embodiment, doxorubicin is administered at a dose of 60 mg/m² in combination with LIV1-ADC and a PD-1 antagonist. In combination with LIV1-ADC and a PD-1 antagonist, paclitaxel is administered at a dose between 50 mg/m² and 300 mg/m². Other appropriate dose ranges of paclitaxel in the combination are 100 mg/m² to 260 mg/m², and 135 mg/m² to 175 mg/m². In an embodiment, paclitaxel is administered at a dose of 175 mg/m² in combination with LIV1-ADC and a PD-1 antagonist. In another embodiment, paclitaxel is administered at a dose of 80 mg/m² in combination with LIV1-ADC and a PD-1 antagonist.

Examples of cancers associated with LIV-1 expression and amenable to treatment with LIV-1-ADC or the combination therapies of the disclosure include breast cancer, prostate cancer, ovarian cancer, endometrial cancer, pancreatic cancer, cervical, liver, gastric, kidney, and squamous cell carcinomas (e.g., bladder, head, neck and lung), skin cancers, e.g., melanoma, small lung cell carcinoma or lung carcinoid, non-small cell lung carcinoma (NSCLC)-squamous or non-squamous, esophagogastric junction (GEJ) adenocarcinoma, esophageal squamous carcinoma, and head & neck squamous carcinoma. The treatment can be applied to patients having primary or metastatic tumors of these kinds. The treatment can also be applied to patients who are refractory to conventional treatments (e.g., for breast cancer: hormones, tamoxifen, HERCEPTIN®), or who have relapsed following a response to such treatments. The methods can also be used on triple negative breast cancers. A triple negative breast cancer is a term of art for a cancer lacking detectable estrogen and progesterone receptors and lacking overexpression of HER2/neu when stained with an anti-body to any of these receptors, such as described in the examples. The methods can also be used on triple positive breast cancers, hormone receptor positive breast cancers, and HER2 positive breast cancers. Staining can be performed relative to an irrelevant control antibody and lack of expression shown from a background level of straining the same or similar to that of the control within experimental error. Likewise, lack of overexpression is shown by staining at the same or similar level within experimental error of noncancerous breast tissue, preferably obtained from the same patient. Alternatively or additionally, triple negative breast cancers are characterized by lack of responsiveness to hormones interacting with these receptors, aggressive behavior and a distinct pattern of metastasis.

In some embodiments, LIV-1-ADC and a PD-1 antagonist are administered in such a way that the combination provides a synergistic or additive effect in the treatment of LIV-1-associated cancer in a patient. Administration can be by any suitable means provided that the administration provides the desired therapeutic effect. In preferred embodiments, LIV-1-ADC and a PD-1 antagonist are administered during the same cycle of therapy, e.g., during one cycle of therapy, e.g., a three or four week time period.

Administration of LIV-1-ADC and PD-1 antagonist can be parenteral, intravenous, oral, subcutaneous, intra-arterial, intracranial, intrathecal, intraperitoneal, topical, intranasal or intramuscular. In various embodiments, LIV-1-ADC is administered by intraperitoneal injection. In another embodiment, LIV-1-ADC is administered by intravenous injection. It is contemplated that the PD-1 antagonist, e.g., an anti-PD-1 antibody such as pembrolizumab, is administered intravenously or subcutaneously. Administration can also be localized directly into a tumor. Administration into the systemic circulation by intravenous or subcutaneous administration is preferred. Intravenous administration can be, for example, by infusion over a period such as 30-90 min or by a single bolus injection.

The frequency of administration can be weekly, bi-weekly, every three weeks, monthly, quarterly, or at irregular intervals in response to changes in the patient's condition or progression of the cancer being treated. In an embodiment, one or both agents of the combination is administered once every three weeks. In another embodiment, one or both agents of the combination is administered once every four weeks. For subcutaneous administration, an exemplary dosing frequency is daily to monthly, although more or less frequent dosing is also possible.

In various embodiments, the LIV1-ADC and PD-1 antagonist therapy is administered for at least 3 cycles, and up to 6, 8, or 10 cycles, for example from 3 to 6 cycles, or 3 to 8 cycles, or for 3, 4, 5, 6, 7, 8, 9 or 10 cycles. In various embodiments, the cycle is a three week cycle.

In various embodiments, LIV-1-ADC, e.g., ladiratuzumab vedotin, therapy is administered by intravenous infusion over the course of about 30 minutes. In various embodiments, anti-PD-1 antibody is administered by intravenous infusion over the course of about 30 minutes or about 60 minutes.

In various embodiments, the disclosure provides a method of treating a subject having unresectable locally-advanced or metastatic (LA/M) triple negative breast cancer (TNBC) who have not previously received cytotoxic therapy comprising administering an effective amount of a composition comprising ladiratuzumab vedotin (A) and an anti-PD-1 antibody, wherein the ladiratuzumab vedotin is administered at 2.0 or 2.5 mg/kg, anti-PD-1 antibody is administered at 100-300 mg/dose, and optionally, wherein the ladiratuzumab vedotin is administered within 30 minutes or 1 hour of the anti-PD-1 therapy. In various embodiments, the LIV-1-ADC is administered once every 3 weeks. In various embodiments, the LIV-1-ADC is administered once weekly. In various embodiments, the anti-PD-1 therapy is administered once every 3 weeks.

In various embodiments, the disclosure provides a method of treating a subject having unresectable locally-advanced or metastatic (LA/M) triple negative breast cancer (TNBC) who has not previously received cytotoxic therapy comprising administering an effective amount of a composition comprising ladiratuzumab vedotin (A) and an anti-PD-1 antibody, wherein the ladiratuzumab vedotin is administered at 1.0 mg/kg, 1.25 mg/kg or 1.75 mg/kg, anti-PD-1 antibody is administered at 100-300 mg/dose, and optionally, wherein the ladiratuzumab vedotin is administered within 30 minutes or 1 hour of the anti-PD-1 therapy. In various embodiments, the LIV-1-ADC is administered once weekly. In various embodiments, the LIV-1-ADC is administered once every 3 weeks. In various embodiments, the anti-PD-1 therapy is administered once every 3 weeks.

In various embodiments, the LIV-1 ADC is administered on Days 1, 8, and 15 of a three week cycle, and pembrolizumab is administered Day 1 of each three week cycle. In various embodiments, the anti-LIV-1 antibody in a LIV-1-ADC is an hLIV22 antibody.

In various embodiments, it is contemplated that the LIV-1 ADC is administered at a dose of 1.0, 1.25, 1.5, 1.75 mg/kg, 2.0 or 2.5 mg/kg to subjects having solid tumors such as non-small cell lung carcinoma (NSCLC) (squamous & non-squamous), small cell lung cancer, gastric adenocarcinoma, esophagogastric junction (GEJ) adenocarcinoma, esophageal squamous cell carcinoma, and head & neck squamous cell carcinoma. Optionally, treatment is administered with a PD-1 antagonist, such as an anti-PD-1 antibody as described herein. The LIV-1-ADC can be administered weekly or every three weeks.

It is contemplated that the methods herein reduce tumor size and/or tumor burden in the subject, and/or reduce metastasis in the subject.

In various embodiments, the methods reduce the tumor size by about 10%, 20%, 30% or more. In various embodiments, the methods reduce tumor size by about 10%, 15%, 20%, 25%, 30%, 35%, 40%, 45%, 50%, 55%, 60%, 65%, 70%, 75%, 80%, 85%, 90%, 95% or 100%.

In various embodiments, the methods reduce the ability of the tumor to grow and lead to stable disease as defined by standard methodologies in the field including RECIST (Response Evaluation Criteria In Solid Tumors) and irRC (immune response criteria).

Effects of LIV-1 ADC or LIV1-ADC+PD-1 antagonist, e.g., anti-PD-1 antibody, on the tumor microenvironment (TME), immune cell infiltrate, cytokine production, and mRNA levels are also measured. These biomarkers are measured using immunohistochemistry, mRNA analysis using GeneSeq or another method known in the art, ELISA, FACS analysis, and other procedures available in the art.

Analysis of the tumor microenvironment can include measurement of immune cells in or around the tumor site, e.g., macrophages, monocytes, dendritic cells (DC) (including conventional DC), Natural killer (NK) cells, CD4+ T cells, CD8+ T cells, regulatory CD4+ T cells, effector memory CD4+ T cells, regulatory CD8+ T cells, and/or PD-L1+ cells. Tumor microenvironment is also analyzed for change in extracellular matrix proteins and/or extracellular remodeling at or near the tumor site before and after treatment, including collagens (collagens I, II, III, V, IX, and XI), heparan sulfate proteoglycans, and fibronectin. Analysis of the tumor microenvironment can also include the measurement of cytokines and chemokines.

Levels of MHC expressed on cells in the tumor, co-stimulatory molecules (CD80 and CD86), and cytokine and chemokine production are also measured before and after treatment with LIV-1-ADC or LIV-1-ADC and PD-1 antagonist, e.g., anti-PD-1 antibody. Cytokines include IFN-γ, TNF-α, TGF-β, IL-2, IL-4, IL-5, IL-13, IL-17, IL-18, IL-21, IL-22, IL-23, IL-32, and IL-33. Chemokines include CXCL9, CXCL10, CXCL11, CXCL12.

Genes that are differentially expressed before and after treatment are also determined, and their correlation with efficacy can also be determined. For example, genes associated with immune cells or immune cell function may be differentially expressed, e.g., SIGLEC1, MS4A4A, CD163, CXCL12, and/or APOE. In various embodiments, the immune activation genes are a HLA-DMA, HLA-DOA and IL-18.

Tumor associated antigens may also be differentially expressed as a result of the treatment described herein.

### Formulations

Various delivery systems can be used to administer antibodies or antibody-drug conjugates contemplated herein. In certain embodiments, administration of the antibody-drug conjugate compound is by intravenous infusion. In some embodiments, administration is by a 30 minute, 1 hour, 90 minute or two hour intravenous infusion. In various embodiments, administration of the antibody compound is by intravenous infusion. In various embodiments, administration is by a 30 minute, 1 hour, 90 minute or two hour intravenous infusion.

The antibody and/or antibody-drug conjugate compound can be administered as a pharmaceutical composition comprising one or more pharmaceutically compatible ingredients. For example, the pharmaceutical composition typically includes one or more pharmaceutically acceptable carriers, for example, water-based carriers (e.g., sterile liquids). Water is a more typical carrier when the pharmaceutical composition is administered intravenously.

The composition, if desired, can also contain, for example, saline salts, buffers, salts, nonionic detergents, and/or sugars. Examples of suitable pharmaceutical carriers are described in “Remington® Pharmaceutical Sciences” by E. W. Martin. The formulations correspond to the mode of administration.

The present disclosure provides, for example, pharmaceutical compositions comprising a therapeutically effective amount of the antibody-drug conjugate, a buffering agent, optionally a cryoprotectant, optionally a bulking agent, optionally a salt, and optionally a surfactant. Additional agents can be added to the composition. A single agent can serve multiple functions. For example, a sugar, such as trehalose, can act as both a cryoprotectant and a bulking agent. Any suitable pharmaceutically acceptable buffering agents, surfactants, cyroprotectants and bulking agents can be used in accordance with the present disclosure.

Pharmaceutical compositions of the present disclosure containing the ADC or antibodies described herein as an active ingredient may contain pharmaceutically acceptable carriers or additives depending on the route of administration. Examples of such carriers or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used are chosen from, but not limited to, the above or combinations thereof, as appropriate, depending on the dosage form of the present disclosure.

Formulation of the pharmaceutical composition will vary according to the route of administration selected (e.g., solution, emulsion). An appropriate composition comprising the antibody or ADC to be administered can be prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers.

A variety of aqueous carriers, e.g., sterile phosphate buffered saline solutions, bacteriostatic water, water, buffered water, 0.4% saline, 0.3% glycine, and the like, and may include other proteins for enhanced stability, such as albumin, lipoprotein, globulin, etc., subjected to mild chemical modifications or the like.

Therapeutic formulations of the antibodies are prepared for storage by mixing the ADC or antibody having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In various embodiments, the antibody drug conjugate formulations including drug conjugate formulations have undergone lyophilization, or other methods of protein preservation, as well as antibody drug formulations that have not undergone lyophilization

The formulations to be used for in vivo administration must be sterile. This is readily accomplished by filtration through sterile filtration membranes. The pharmaceutical compositions may be in the form of a sterile injectable aqueous, oleaginous suspension, dispersions or sterile powders for the extemporaneous preparation of sterile injectable solutions or dispersions. The suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. The carrier can be a solvent or dispersion medium containing, for example, water, ethanol, polyol (for example, glycerol, propylene glycol, and liquid polyethylene glycol, and the like), suitable mixtures thereof, vegetable oils, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Aqueous suspensions may contain the active compound in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate.

### Kits

The present disclosure also provides kits for the treatment of a LIV-1 expressing cancer. The kit can comprise (a) a container containing the antibody-drug conjugate and optionally, containers comprising one or more of PD-1 antagonist, such as an anti-PD-1 antibody, and optionally additional chemotherapeutics. Such kits can further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, can also be included in the kit.

### Example 1

LV-induced immunogenic cell death (ICD) elicits an inflammatory response and increases tumor immune cell infiltration (Schmid et al., N Engl J Med. 2018; 379(22): 2108-21). It is hypothesized that LV-induced ICD creates a microenvironment favorable for enhanced checkpoint inhibitor activity. In order to determine if LV and PD-1 antagonist combination therapy is safe and effective, a single-arm, open label, phase 1b/2 study combining LV+ pembrolizumab was carried out in patients with unresectable locally-advanced or metastatic (LA/M) triple negative breast cancer (TNBC) who have not previously received cytotoxic therapy for their advanced disease.

In Part A, a dose finding study was undertaken to determine doses of LV and pembrolizumab that may be safe when combined. Patients were administered LV at either 2.5 mg/kg or 2.0 mg/kg in combination with pembrolizumab at 200 mg/kg weekly for three weeks (Q3wk). In Part B, additional patients were enrolled in the expansion phase and the same administration regimen followed.

Patients were treated if the following criteria were met:
Metastatic or locally-advanced, histologically documented TNBC (absence of HER2, ER, and PR expression per 2014 ASCO guideline)
No prior cytotoxic therapy for the treatment of unresectable LA/M BC
No prior anti-PD(L)1 therapy
≥6 months since the latest curative (neo)adjuvant treatment
Measurable disease per RECIST v1.1
ECOG of 0 or 1
Meets baseline laboratory test criteria
Able to provide tissue samples for biomarker analysis
Neuropathy Grade (Gr) 2
Adequately treated CNS metastases and off corticosteroids

Age, gender, and ECOG are generally consistent with TNBC populations in other studies. Fewer subjects were enrolled with de novo MTNBC compared to recently published randomized studies in frontline TNBC (Kim et al., Lancet Oncol. 2017; 18(10): 1360-1372; Yardley et al., Annals of Oncology. 2018; 19: 1763-1770; Brufsky et al., J Clin Oncol 37(Suppl 15): Abstract 1013; Cortes et al., Ann Oncol 30(Suppl 5): v859-60). Subjects were excluded if they had undergone prior immune-oncology therapy, had pre-existing neuropathy of at least Grade 2, had active central nervous system (CNS) metastases, and/or experienced active autoimmune disease requiring systemic treatment within the past 2 years.

TABLE 2
Subject characteristics

| | N = 77^{a} |
|---|---|
| Median age in years, (min, max) | 56 (30-82) |
| Female gender, n (%) | 77 (100%) |
| ECOG performance status, n (%) | |
| 0 | 37 (48%) |
| 1 | 40 (52%) |
| de novo TNBC, n (%) | 17 (22%) |
| Interval between (neo) adjuvant and 1L therapy, n (%) | |
| >12 months | 40 (52%) |
| 6-12 months | 15 (19%) |
| Tumor histology, n (%) | |
| Invasive ductal carcinoma | 52 (68%) |
| Invasive lobular carcinoma | 4 (5%) |
| Others | 21 (53%) |
| Liver metastasis at baseline, n (%) | 16 (21%) |
| Lung metastasis at baseline, n (%) | 41 (53%) |

Antibody drug conjugate LV was administered at 2.5 or 2.0 mg/kg IV every 3 weeks over approximately 30 minutes. Pembrolizumab was administered at 200 mg IV every 3 weeks over approximately 30 minutes. Prophylactic G-CSF was required when receiving LV 200 mg per cycle. Patients were analyzed by tumor biopsy at baseline (Parts A and B), by C1D5 expression (Part A), and C1D15 expression (Part B).

Primary endpoints for the study included objective response rate (ORR) RECIST v1.1 and safety. Secondary Endpoints include duration of response (DOR), progression free survival (PFS), overall survival (OS), and PK/PD of the antibodies.

In Part A, no subject (0/7) experienced dose limiting toxicity (DLT) at 2.0 mg/kg LV and 200 mg pembrolizumab, and 2/12 subjects experienced a DLT at 2.5 mg/kg LV and 200 mg pembrolizumab, exhibiting either Grade 3 colitis or Grade 3 diarrhea.

Adverse Events: Treatment related adverse events were minimal, with no new safety signals observed with the combination. Most AEs were low to moderate (Grade 1-2) in severity, and included nausea, fatigue, and alopecia (Table 3). The most common severe (Grade 3-4) AE was neutropenia (14%), and most common serious AEs were abdominal pain (5%), colitis (4%), and pyrexia (4%). The most common AEs attributed to pembrolizumab in the combination were maculo-papular rash (9%), diarrhea (6%), colitis (4%), and pruritis (4%), which were similar in frequency to that of pembrolizumab monotherapy (USPI). There were no Grade 5 treatment-related AEs.

TABLE 3
Treatment-related AEs in >20% of Patients

| | Total | |
|---|---|---|
| | (N = 77)^{b} | |
| Preferred Term | All grades | Gr ≥ 3^{c} |
| Nausea | 45 (58%) | 5 (6%) |
| Fatigue | 40 (52%) | 2 (3%) |
| Alopecia | 38 (49%)^{d} | N/A |
| Diarrhea | 31 (40%) | 4 (5%) |
| Decreased appetite | 25 (32%) | 1 (1%) |
| Peripheral sensory neuropathy | 23 (30%) | 4 (5%) |
| Vomiting | 17 (22%) | 2 (3%) |

^{a}Related to either LV or pembrolizumab per the investigator
^{b}LV 2.5 mg/kg: n = 59; LV 2.0 mg/kg: n = 18
^{c}No Grade 4 events for the listed AEs
^{d}Gr2 alopecia: 27%

The most common AEs leading to LV dose reduction were peripheral neuropathy (9%) and abdominal pain (3%). The most common AE leading to dose discontinuation was peripheral neuropathy (3%). Discontinuation rate for AEs was 14% for LV and 13% for pembrolizumab (Table 4)

TABLE 4
Reduction and Discontinuation Rates

| | | Discontinuation |
|---|---|---|
| | | of LV and |
| | Reduction^{a} | pembro |
| LV 2.5 or 2.0 mg/kg + | 21 (27% | 8 (10%) |
| Pembrolizumab (N = 77), n (%) | | |

^{a}Dose reduction applies to LV only

The results show that LV+pembrolizumab achieved a confirmed objective response rate of 35% as measured by RECIST v1.1. Stable disease was observed in 32 subjects (48%). Of the test population, 4 subjects (6%) had a partial response (PR) or complete response (CR) awaiting a confirmatory scan, 10 subjects (15%) had a best response of PR not confirmed (8 off treatment due to progressive disease, 2 off treatment due to AEs), and 18 subjects (27%) had SD. Disease progression was reported as the best response for 8 subjects (12%), with the best overall response (confirmed and unconfirmed) was 56% (Table 5).

TABLE 5
Response Rates

| | Total | |
|---|---|---|
| | (N = 66)^{a} | |
| Confirmed ORR, % (95% CI)^{b} | 35% | (23.5, 47.6) |
| CR confirmed | 2 | (3%) |
| PR confirmed | 21 | (32%) |
| SD | 32 | (48%) |
| PD | 8 | (12%) |
| Not evaluable/not done | 3 | (5%) |

^{a}All subjects whose C1D1 date was at least 3 months before the data extract
^{b}Best overall response is the proportion of subjects who achieved either a confirmed or unconfirmed PR or CR per investigator assessment using RECIST v1.1. Subjects without any post-baseline tumor assessments are considered non-responders

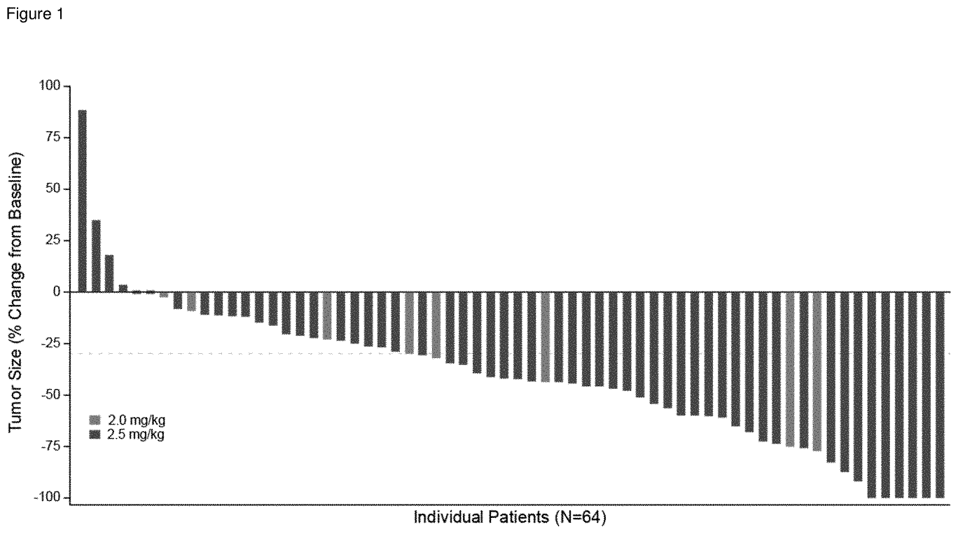

Analysis of tumor burden after treatment shows that >90% of subjects achieved tumor reduction (FIG. 1). The efficacy evaluable population includes all treated subjects with at least one evaluable post-baseline assessment according to RECIST v1.1 or who had discontinued from the study (N=69). Of the efficacy evaluable population, 5 subjects did not have evaluable response assessments before study discontinuation.

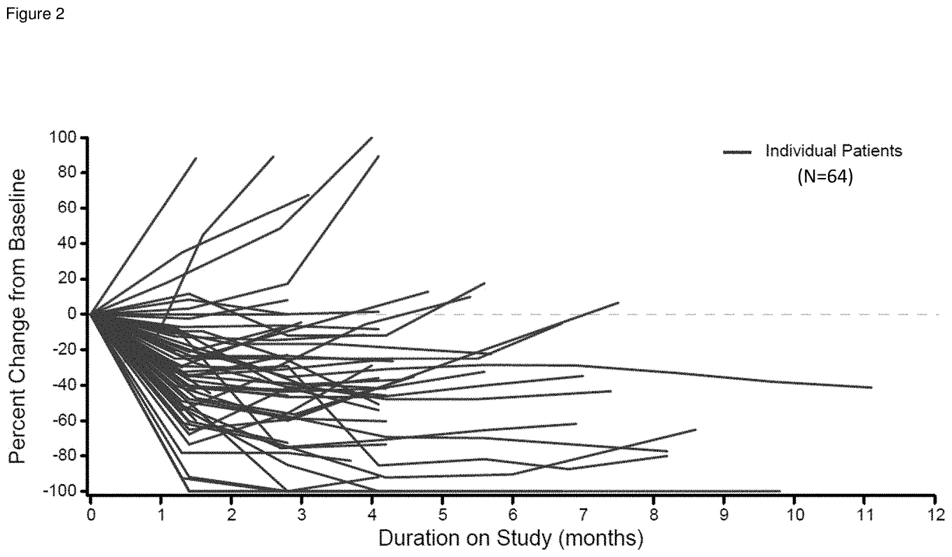

Tumor burden was measured based on change in tumor size over time of treatment. 45% of subjects achieved response at the 1^{st} assessment. The median time to the 1^{st} response was 1.4 months, with a median duration of response of 4.4 months (min, max: 1.4+, 8.4+), and a median follow-up time of 3.7 months (FIG. 2).

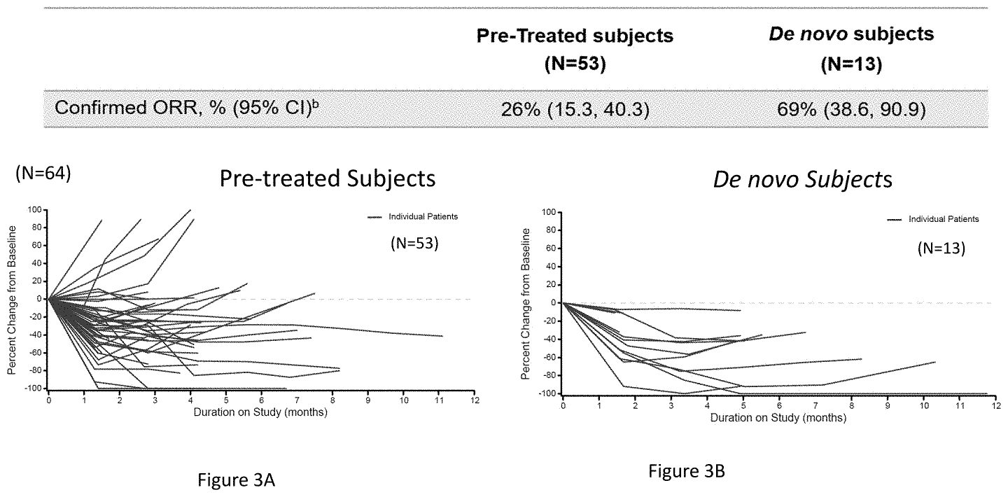

Subjects with de novo first line (1L) metastatic triple negative breast cancer (MTNBC) account for 22% of the study population. ORR was higher in de novo vs. in pre-treated subjects, 69% to 26%, respectively, and a majority of de novo patients achieved durable tumor control starting at 5 months up to 10 months (FIG. 3).

Levels of PD-L1 on the tumors were assessed. 38 tumor samples yielded results for PD-L1 status (Dako 22C3), with 35% PD-L1 positive and 40% of those sampled PD-L1 negative. Frequency of PD-L1 positivity is in line with recently reported pembrolizumab TNBC studies (Schmid et al., Ann Oncol 30(Suppl 5): v853-4). Responses were observed consistently across PD-L1 status. Preliminary analysis showed no difference in response rates based on LIV-1 expression (Table 6).

TABLE 6
Response Rate Based on PD-L1 Expression

| | PD-L1 | PD-L1- | |
|---|---|---|---|
| | positive | negative | PD-L1 |
| | (CPS ≥ 1) | (CPS < 1) | unknown |
| | (N = 23) | (N = 15) | (N = 28) |
| Confirmed ORR % | 35% | 40% | 32% |
| (95% CI) | (16.4, 57.3) | (16.3, 67.7) | (15.9, 52.4) |

Conclusions: LV in combination with pembrolizumab is a safe and tolerable first-line therapy in MTNBC. Both LV and pembrolizumab can be delivered at the doses used in single-agent trials. No new safety signals were observed with the combination and the AE profile was tolerable and manageable.

Efficacy of the combination is encouraging, showing that the ORR was 35% in the study population, and 69% in subjects with de novo MTNBC. Over 90% of subjects achieved tumor reduction, and 45% of responses occurred at the 1st response assessment. A small minority (12%) of subjects had disease progression as their best response. However, duration of treatment in de novo MTNBC subjects is promising and surprising. Biomarker assessment showed that response rate was consistent regardless of PD-L1 status. Subjects continue to be followed for duration of response, PFS, and OS.

### Example 2

In order to enhance efficacy and improve the tolerability profile, LV delivered weekly with pembrolizumab is being evaluated. A single-arm, open-label Phase 1b/2 study of LV in combination with pembrolizumab as first line therapy for patients with unresectable locally advanced or mTNBC is conducted (CT.gov: NCT03310957, EudraCT: 2017-002289-35) to evaluate the safety and efficacy of LV at either 1.00 or 1.25 mg/kg/week on Days 1, 8, and 15+pembrolizumab 200 mg on Day 1 of each cycle. Approximately 24 patients are enrolled in the LV weekly cohorts. Patients must not have had prior cytotoxic or anti-PD(L)1 treatment for advanced disease, have measurable disease per RECIST v1.1, and an ECOG score of 0 or 1.

The primary objectives are to evaluate the safety/tolerability and objective response rate of weekly LV+pembrolizumab. The secondary objectives include evaluation of DOR, DCR, PFS, and OS.

### Example 3

LIV-1 is Involved in signaling that promotes cancer cells to metastasize (Lue et al, PLOS One, 6: e27720; 2011), and clinically, LIV-1 expression in tumors linked with progression to metastasis (Manning et al, EJC, 5: 675-678; 1994). A Phase 2 study is undertaken to evaluate the effects of LIV-1-ADC in solid tumors such as non-small cell lung carcinoma (NSCLC) (Squamous & Non-Squamous), small cell lung cancer, gastric/GEJ adenocarcinoma, esophageal squamous carcinoma, and head & neck squamous carcinoma.

In a Phase 2, global single arm trial subjects having unresectable locally advanced/metastatic solid tumors, such as small cell lung cancer, or NSCLC, squamous or non-squamous, are treated with 2.5 mg/kg LIV1-ADC every three weeks, or in a dosing study are treated with various doses of LIV1-ADC to determine therapeutic safety, e.g., 1.0 mg/kg or 1.25 mg/kg administered weekly.

Eligible patients having small cell lung cancer will exhibit: extensive disease stage, measurable disease per RECIST 1.1, line of prior platinum-based chemo for extensive disease stage; received prior anti-PD(L)1 therapy if eligible, neuropathy ≤Gr2, and patients with mixed SCLC/neuroendocrine with NSCLC histology are not eligible.

Eligible patients having NSCLC, e.g., squamous or nonsquamous NSCLC, will exhibit: unresectable locally advanced or metastatic disease, measurable disease per RECIST 1.1, ≤1 line of prior platinum-based chemo for the advanced disease, progression during or following chemo for advanced disease, or within 6 mos of last dose of chemo with curative intent, treated for actionable mutations excluded, received prior anti-PD(L)1 therapy unless contraindicated, and exhibit neuropathy ≤Gr2.

Primary endpoints include objective response rate (ORR) while secondary endpoints include safety, DoR, PFS and OS. Biomarkers are also evaluated for changes as a result of treatment with LIV1-ADC.

### Example 4

To determine the effects of treatment with LIV1-ADC and anti-PD-1 antibody on cellular activity and gene expression in tumors, a correlative biomarker study was undertaken to assess the ability of LIV-1-ADC (LV) to modulate the tumor microenvironment (TME) in triple negative breast cancer (TNBC) patients.

Patients with locally advanced or metastatic TNBC were enrolled in LVA-001 (LV monotherapy, 2˜2.5 mg/kg, every 3 weeks [q3w]) and LVA-002 [LV plus pembro (anti-PD-1) 200 mg, q3w]. Tumor biopsies were collected at baseline and after the first dose of treatment (cycle 1 day 5 (C1D5) in LVA-001; C1D5 or C1D15 in LVA-002. RNA extraction and RNAseq was performed to examiner gene expression before and after treatment. Differential gene expression was assessed using the DESeq2 package (Love et al., (2014) Genome Biology, 15: 550). Gene ontology (GO) analysis was carried out using the TopGO R package (Alexa A, Rahnenfuhrer J (2020). topGO: Enrichment Analysis for Gene Ontology. R package version 2.40.0) using a Fisher test in conjunction with the “weight” scoring algorithm to assess significantly enriched biological process nodes. Scoring of potential cell type enrichment was done using the xCell method (Aran, et al., Genome Biology (2017) 18:220) implemented in R. Unless otherwise noted, false discovery rates (FDRs) for individual gene expression comparisons were calculated by the Benjamini-Hochberg method and DESeq p-values. Immunohistochemistry (IHC) evaluation were performed to measure levels of CD8, CD68 and PD-L1 (22C3) in subjects. Combined Positive Score (CPS) was scored according to Agilent's PD-L1 IHC 22C3 pharmDx interpretation manual.

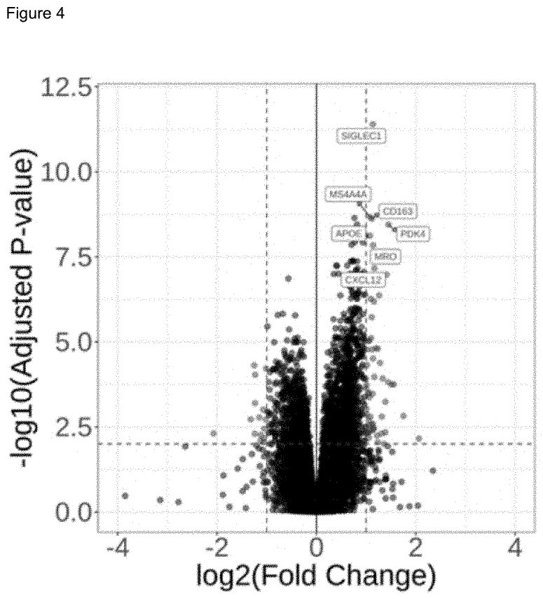

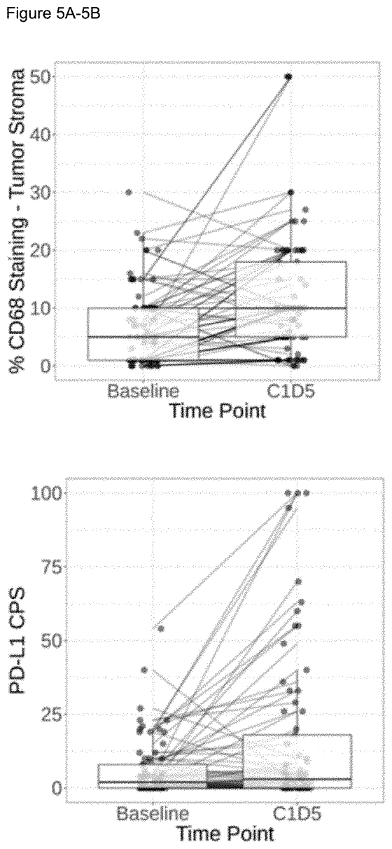

Paired baseline and on-treatment (C1D5) biopsies from LVA-001 (LV monotherapy) were used for RNAseq (N=59). Differential expression was assessed by comparing paired baseline-C1D5 RNAseq profiles. Differentially expressed genes (FIG. 4) were identified using fold-change and significance cutoffs (absolute fold change >2, adjusted p-value/FDR<0.01). 59 up-regulated genes were identified at C1D5 and 15 down-regulated genes at C1D5. Some of the top differentially expressed genes (SIGLEC1, MS4A4A, CD163) relate to macrophage function. Genes differentially expressed at an FDR<1e-7 are shown with labels in FIG. 4 and include macrophage markers such as SIGLEC1, CD163 and MS4A4A, as well as APOE, CXCL12, MRO, and PDK4.

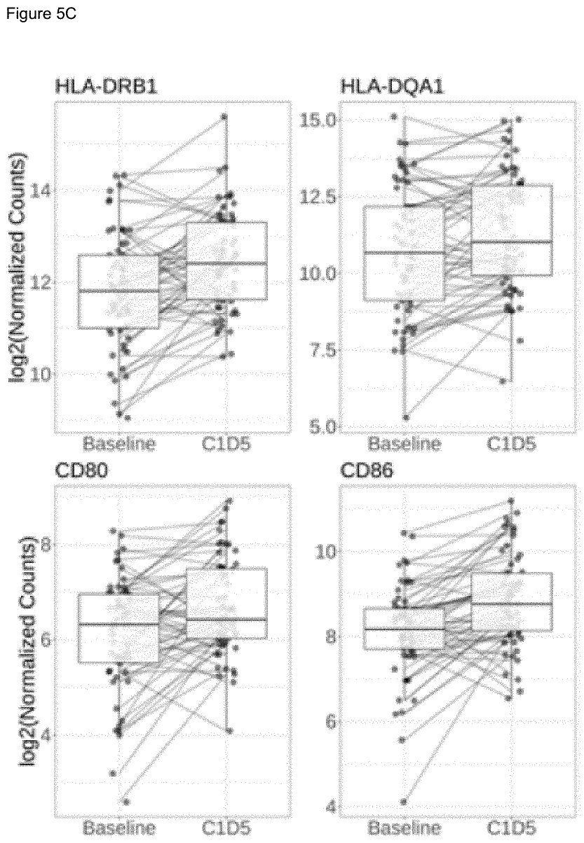

Analysis shows that LV therapy increases macrophage infiltration, induces MHC, co-stimulatory molecules, and PD-L1 expression. IHC was carried out to determine macrophage (CD68) and PD-L1 (22C3) staining in tumor biopsies at baseline and C1D5 of an LVA-001 patient. FIG. 5A-5B illustrates CD68 IHC results (% of cells positive for CD68 in tumor stroma) and PD-L1 CPS scores in all LVA-001 patients with evaluable samples (N=79 for CD68; N=73 for PD-L1), showing that treated patients have higher levels of CD68 and PD-L1 staining in the tumor. FIG. 5C shows induction of MHCII genes (HLA-DRB1 and HLA-DQA1) and co-stimulatory molecules (CD80 and CD86) by RNAseq analysis (N=59), each of which appear to increase in treated subjects. There was also significant induction of MHCI genes, inflammatory cytokines, and overall increase in tumor inflammation score. These results demonstrate that LV monotherapy induced immune activation in TME in breast cancer patients.

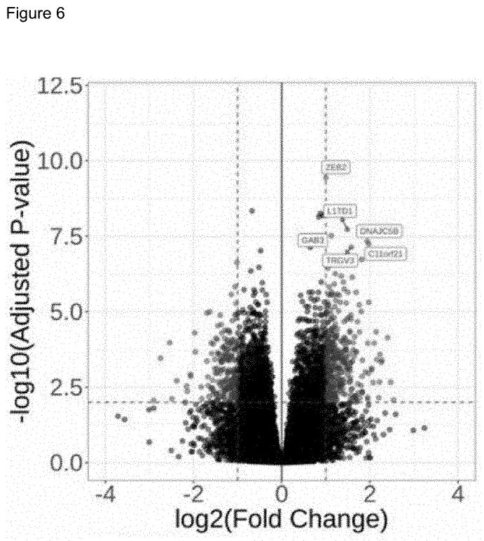

The effects of LV and pembrolizumab on immune activation in TME breast cancer patients was also assessed in LVA-002. Paired baseline and on-treatment (C1D15) from initial data set were used for RNAseq (N=16). Differential gene expression was assessed by comparing paired baseline C1D15 RNAseq profiles (FIG. 6). Differentially expressed genes were identified using fold-change and significance cutoffs (absolute fold-change >2, adjusted p-value/FDR <0.01). 342 upregulated genes were identified at C1 D15 and 192 down-regulated genes at C1D15. ZEB2, L1TD1, DNAJCSB, GAB3, C11orf21 and TRGV3 were upregulated at an FDR<1e-7 (FIG. 6, shown with labels). IHC analysis showed increased infiltration of CD3+ and CD8+ T cells, CD68+ macrophages, and increased PD-L1 staining in C1D5 biopsy as compared to baseline biopsy collected from an LVA-002 patient.

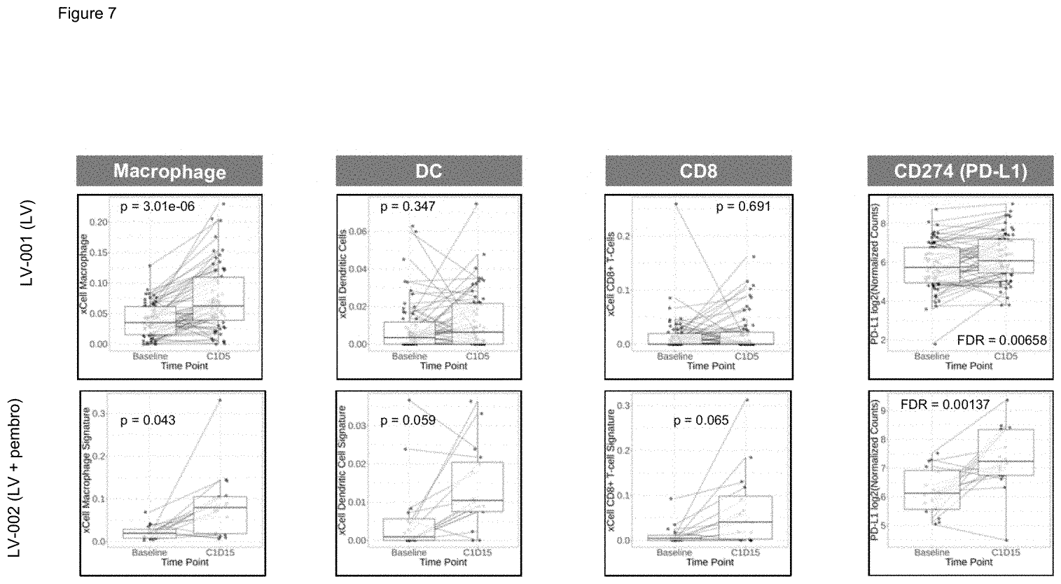

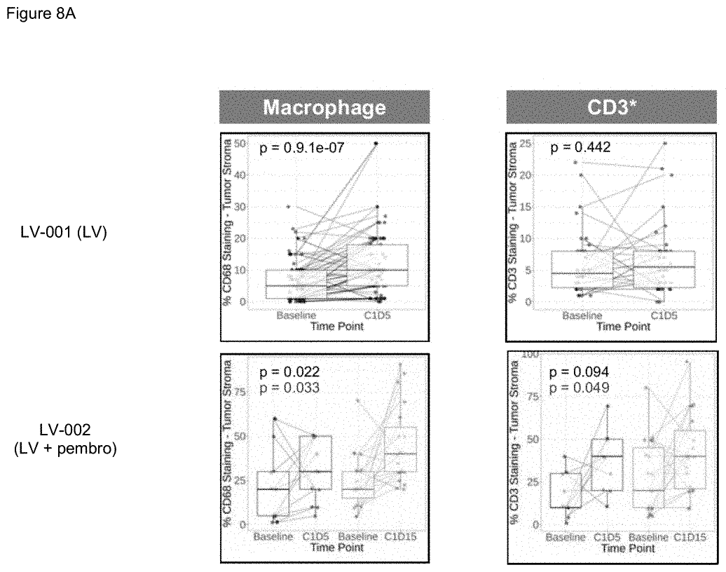

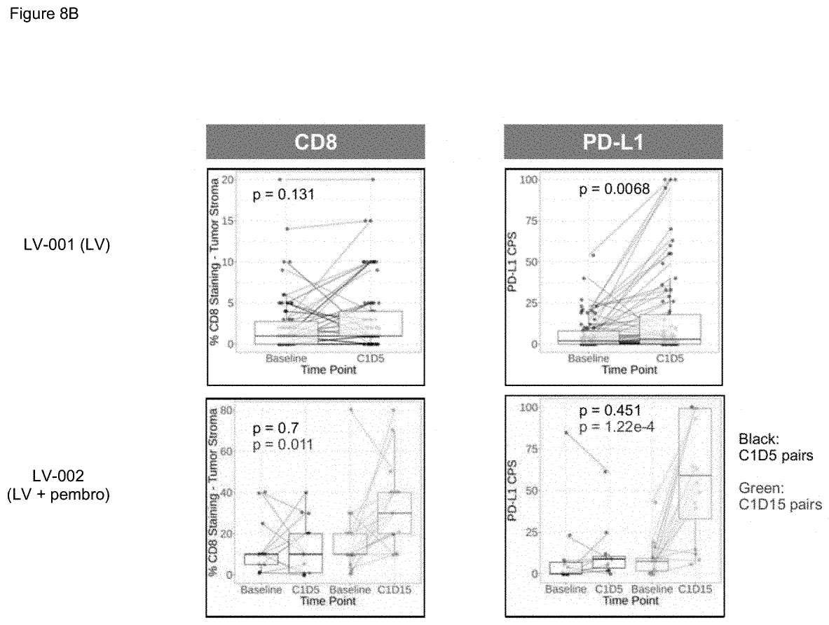

Further analysis of cell biomarkers was performed by comparing RNA levels and immune cell infiltrate by immunohistochemistry. A comparison of RNA levels on tumor infiltrating immune cells (macrophage, DC, and CD8 T cells) and CD274 (PD-L1) gene expression in LVA-001 (LV alone) and LVA-002 (LV+pembro) shows an increase in macrophages and CD8 T cells in treated subjects compared to baseline (FIG. 7). For PD-L1, the FDR is derived from the differential expression analysis comparing C1D5/C1D15 and baseline. N=59 for LVA-001; N=16 for LVA-002. This shows a stronger induction of DC and CD8 T cell signatures in LV+pembro treated subjects by RNA analysis. Tumor infiltrating immune cell (CD3+ and CD8+ T cells, CD68+) and CD274 (PD-L1) staining in LVA-001 and LVA-002 subjects was also carried out by IHC. On-treatment biopsies were collected around C1D5 in LVA-001 and around C1D5 or C1D15 in LVA-002. FIGS. 8A and 8B show that there is a stronger induction of CD8 TIL in LV+ pembro treatment at C1D15.

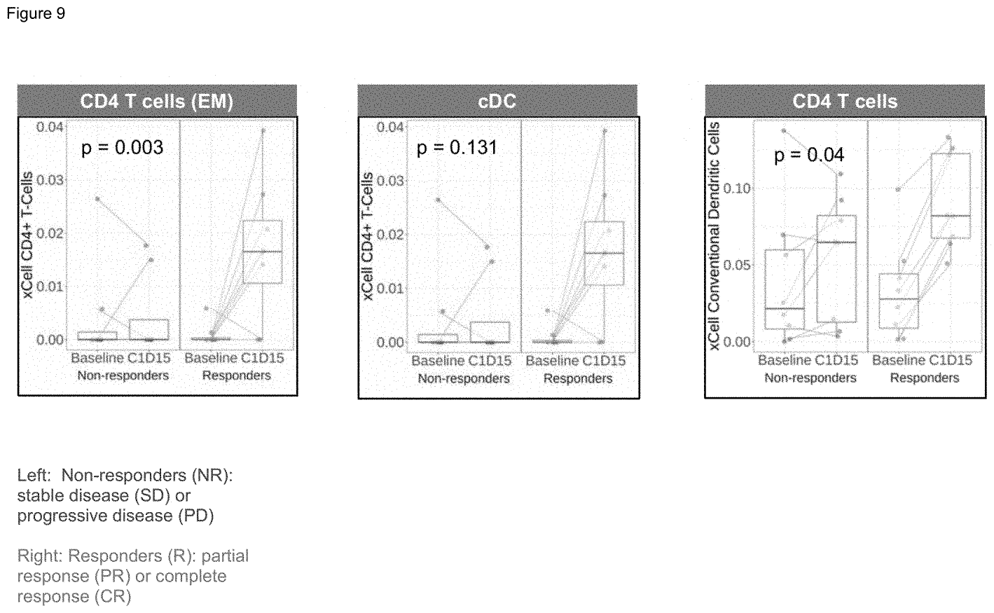

The change in gene signature and cell infiltrate was then assessed for impact on clinical response, for example in subjects characterized as responders, including partial response (PR) or complete response (CR), and non-responders, including those with stable disease (SD) or progressive disease (PD). LV+Pembro treatment-induced an increase in CD4 T cells and DC in tumor are associated with clinical response (LVA-002). xCell gene signature analysis of RNAseq data showed that induction of genes associated with CD4 T cells (especially Effector Memory (EM) CD4 T cells) and conventional DC (cDC) are associated with clinical responses (FIG. 9). The full model considered the xCell score as dependent on response, timepoint, and the interaction of those two features. The simpler, nested, model considered the xCell score as dependent on just response and timepoint. Thus, the resulting likelihood ratio test assessed the importance of the interaction between time and response and the relationship to xCell signature values. A more significant p-value indicates that xCell signatures are behaving differently over time between responders and non-responders.

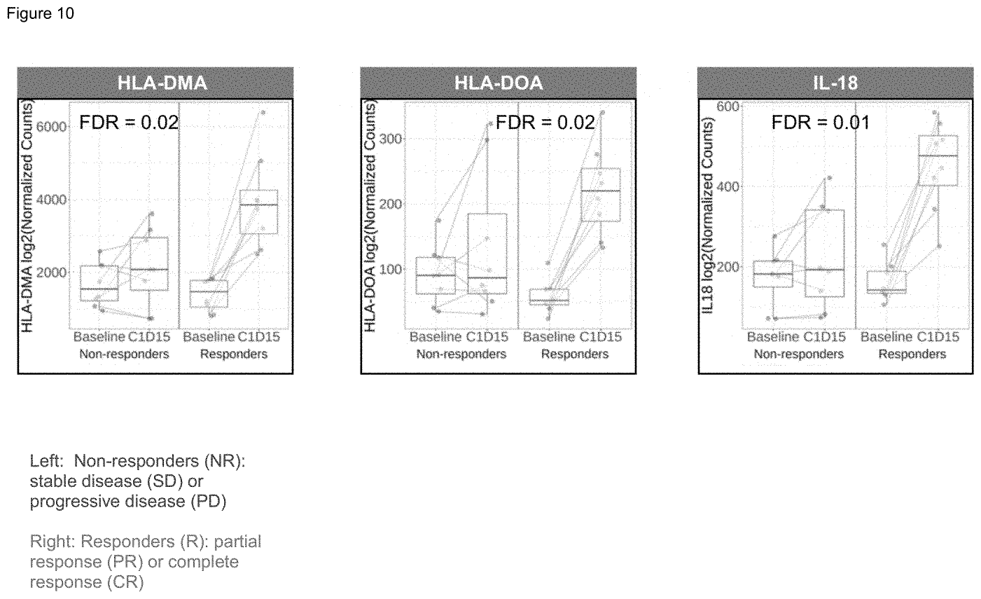

Induction of MHC genes and IL-18 are associated with clinical responses in treated subjects (FIG. 10). The term tested between these two models is the interaction between timepoint and response. Overall 93 genes were observed that are differentially regulated (FDR<0.05) across time between Responders and Non-responders, suggesting that patients with tumors that respond to therapy receiving LV+pembro have greater MHC and inflammatory cytokine induction.

Demonstrated herein is that LV monotherapy results in immune activation in the TME in patients with metastatic TNBC with a significant induction of macrophage infiltration, increase in MHCI/II, co-stimulatory molecules, pro-inflammatory cytokines/chemokines, and PD-L1. LV-induced TME changes in cancer patients are consistent with preclinical evidence that LV induces ICD. The results above suggest that the combination of LV+pembro results in a potent immune activation in the TME with activation of adaptive immune response pathways and increased infiltration of CD8 T cells in addition to macrophages. Preliminary analysis showed that infiltration of T cells and DC, and induction of immune activation genes (MHC and cytokines) are associated with clinical responses in the context of LV and pembro combination therapy.

Numerous modifications and variations of the disclosure as set forth in the above illustrative examples are expected to occur to those skilled in the art. Consequently only such limitations as appear in the appended claims should be placed on the disclosure.

1. A method for treating a subject having or at risk of cancer, the method comprising administering to the subject a LIV-1 antibody drug conjugate (LIV-1-ADC) and a PD-1 antagonist selected from the group consisting of an anti-PD-1 antibody and an anti-PD-L1 antibody.

2. The method of claim 1, wherein the subject has breast cancer, prostate cancer, ovarian cancer, endometrial cancer, pancreatic cancer, lung cancer, a cervical cancer, a melanoma, or squamous cell carcinoma.

3. The method of claim 1, wherein the breast cancer is triple negative breast cancer, triple positive breast cancer, HER2-positive breast cancer, or hormone receptor positive cancer, or wherein the subject has unresectable locally-advanced or metastatic (LA/M) triple negative breast cancer (TNBC).

4-6. (canceled)

7. The method of claim 1 wherein the subject has not previously received cytotoxic therapy.

8. The method of claim 1, wherein the LIV-1-ADC is administered at a dosage between 1.0 mg/kg and 4 mg/kg of the subject's body weight.

9. The method of claim 1, wherein the LIV-1-ADC is administered at a dosage of 1.0 mg/kg of the subject's body weight, at a dosage of 1.25 mg/kg of the subject's body weight, at a dosage of 2.5 mg/kg of the subject's body weight or at a dosage of 2.0 mg/kg of the subject's body weight.

10-12. (canceled)

13. The method of claim 1, wherein the LIV-1-ADC is administered once every 3 weeks or once weekly.

14. (canceled)

15. The method of claim 1, wherein the LIV-1-ADC is administered by intravenous injection.

16. The method of claim 1, wherein the PD-1 antagonist is an anti-PD-1 antibody selected from the group consisting of pembrolizumab or nivolumab.

17. (canceled)

18. The method of claim 1, wherein the PD-1 antagonist is anti-PD-1 antibody pembrolizumab and is administered at a dosage between 100 and 300 mg once every three weeks.

19. The method of claim 1, wherein the anti-PD-1 antibody is administered by intravenous infusion.

20. A method for treating a subject having or at risk of triple negative breast cancer, the method comprising administering to the subject a LIV-1 antibody drug conjugate (LIV-1-ADC) and a PD-1 antagonist selected from the group consisting of an anti-PD-1 antibody and an anti-PD-L1 antibody.

21. The method of claim 20, wherein the subject has unresectable locally-advanced or metastatic (LA/M) triple negative breast cancer (TNBC).

22. The method of claim 20, wherein the subject has not previously received cytotoxic therapy.

23. The method of claim 20, wherein the LIV-1-ADC is administered at a dosage between 1.0 mg/kg and 4.0 mg/kg of the subject's body weight.

24. The method of claim 20, wherein the LIV-1-ADC is administered at a dosage of 1.0 mg/kg of the subject's body weight, a dosage of 1.25 mg/kg of the subject's body weight, at a dosage of 2.5 mg/kg of the subject's body weight or at a dosage of 2.0 mg/kg of the subject's body weight.

25-27. (canceled)

28. The method of claim 20, wherein the LIV-1-ADC is administered once every 3 weeks or once weekly.

29. (canceled)

30. The method of claim 1, wherein the anti-LIV-1 antibody of the LIV-1-ADC is a monoclonal anti-LIV-1 antibody.

31. The method of claim 1, wherein the anti-LIV-1 antibody of the LIV-1-ADC comprises a humanized hLIV22 antibody.

32. The method of claim 1, wherein the anti-LIV-1 antibody of the LIV-1-ADC comprises
i) an amino acid sequence at least 85% identical to a heavy chain variable region set out in SEQ ID NO: 4 and
ii) an amino acid sequence at least 85% identical to a light chain variable region set out in SEQ ID NO: 3.

33. The method of claim 1, wherein the antibody drug conjugate comprises monomethyl auristatin E and a protease-cleavable linker.

34. The method of claim 33, wherein the protease cleavable linker comprises a thiolreactive spacer and a dipeptide, optionally wherein the protease cleavable linker consists of a thiolreactive maleimidocaproyl spacer, a valine-citrulline dipeptide, and a p-amino-benzyloxycarbonyl spacer.

35. (canceled)

36. The method of claim 1, wherein the LIV-1-ADC is ladiratuzumab vedotin.

37. The method of claim 1, wherein (i) the anti-PD-1 antibody cross-competes with nivolumab or pembrolizumab for binding to human PD-1; (ii) the anti-PD-1 antibody binds to the same epitope as nivolumab or pembrolizumab; (iii) the anti-PD-1 antibody is nivolumab; (iv) the anti-PD-1 antibody is pembrolizumab; or (v) the anti-PD-1 antibody is a pembrolizumab variant.

38-39. (canceled)

40. The method of claim 20, wherein the anti-PD-1 antibody is pembrolizumab and is administered at a dosage between 100 and 300 mg once every three weeks.

41-42. (canceled)

43. The method of claim 1, wherein
i) the LIV-1-ADC is ladiratuzumab vedotin and is administered at 2.5 mg/kg, and the anti-PD-1 antibody is pembrolizumab and is administered at a dose of 1-2 mg/kg, or 100-300 mg once every three weeks;
ii) the LIV-1-ADC is ladiratuzumab vedotin and is administered at 2.0 mg/kg, and the anti-PD-1 antibody is pembrolizumab and is administered at a dose of 1-2 mg/kg, or 100-300 mg once every three weeks; or
iii) the LIV-1-ADC is ladiratuzumab vedotin and is administered at 1.0 mg/kg, and the anti-PD-1 antibody is pembrolizumab and is administered at a dose of 1-2 mg/kg, or 100-300 mg once every three weeks.

44-47. (canceled)

48. The method of claim 43, wherein the LIV-1-ADC is administered once every three weeks or once weekly.

49-51. (canceled)

52. A method for treating a subject having de novo metastatic triple negative breast cancer, the method comprising administering to the subject a LIV-1 antibody drug conjugate (LIV-1-ADC) and a PD-1 antagonist selected from the group consisting of an anti-PD-1 antibody and an anti-PD-L1 antibody.

53. The method of claim 52, wherein the subject has not previously received cytotoxic therapy.

54. The method of claim 52, wherein the LIV-1-ADC is administered at a dosage between 1.0 mg/kg and 4.0 mg/kg of the subject's body weight.

55. The method of claim 52, wherein the LIV-1-ADC is administered at a dosage of 1.0 mg/kg of the subject's body weight, a dosage of 1.25 mg/kg of the subject's body weight, at a dosage of 2.5 mg/kg of the subject's body weight or at a dosage of 2.0 mg/kg of the subject's body weight.

56-58. (canceled)

59. The method of claim 52, wherein the LIV-1-ADC is administered once every 3 weeks or once weekly.

60. (canceled)

61. The method of claim 52, wherein the anti-LIV-1 antibody of the LIV-1-ADC is a monoclonal anti-LIV-1 antibody.

62. The method of claim 52, wherein the anti-LIV-1 antibody of the LIV-1-ADC comprises a humanized hLIV22 antibody.

63. The method of claim 52, wherein the anti-LIV-1 antibody of the LIV-1-ADC comprises
i) an amino acid sequence at least 85% identical to a heavy chain variable region set out in SEQ ID NO: 4 and
ii) an amino acid sequence at least 85% identical to a light chain variable region set out in SEQ ID NO: 3.

64. The method of claim 52, wherein the antibody drug conjugate comprises monomethyl auristatin E and a protease-cleavable linker.

65. The method of claim 64, wherein the protease cleavable linker comprises a thiolreactive spacer and a dipeptide, optionally wherein the protease cleavable linker consists of a thiolreactive maleimidocaproyl spacer, a valine-citrulline dipeptide, and a p-amino-benzyloxycarbonyl spacer.

66. (canceled)

67. The method of claim 52, wherein the LIV-1-ADC is ladiratuzumab vedotin.

68. The method of claim 52, wherein (i) the anti-PD-1 antibody cross-competes with nivolumab or pembrolizumab for binding to human PD-1; (ii) the anti-PD-1 antibody binds to the same epitope as nivolumab or pembrolizumab; (iii) the anti-PD-1 antibody is nivolumab; (iv) the anti-PD-1 antibody is pembrolizumab; or (v) the anti-PD-1 antibody is a pembrolizumab variant.

69. (canceled)

70. (canceled)

71. The method of claim 68, wherein the anti-PD-1 antibody is pembrolizumab and is administered at a dosage between 100 and 300 mg once every three weeks.

72-73. (canceled)

74. The method of claim 52, wherein i) the LIV-1-ADC is ladiratuzumab vedotin and is administered at 2.5 mg/kg, and the anti-PD-1 antibody is pembrolizumab and is administered at a dose of 1-2 mg/kg, or 100-300 mg once every three weeks;
ii) the LIV-1-ADC is ladiratuzumab vedotin and is administered at 2.0 mg/kg, and the anti-PD-1 antibody is pembrolizumab and is administered at a dose of 1-2 mg/kg, or 100-300 mg once every three weeks;
iii) the LIV-1-ADC is ladiratuzumab vedotin and is administered at 1.0 mg/kg, and the anti-PD-1 antibody is pembrolizumab and is administered at a dose of 1-2 mg/kg, or 100-300 mg once every three weeks; or
iv) the LIV-1-ADC is ladiratuzumab vedotin and is administered at 1.25 mg/kg, and the anti-PD-1 antibody is pembrolizumab and is administered at a dose of 1-2 mg/kg, or 100-300 mg once every three weeks.

75-78. (canceled)

79. The method of claim 74, wherein the LIV-1-ADC is administered once every three weeks or once weekly.

80-82. (canceled)

83. A method of treating a solid tumor that has metastasized or is at risk of metastasizing comprising administering to the subject a LIV-1 antibody drug conjugate (LIV-1-ADC), wherein the LIV-1-ADC is administered at a dosage between 1.0 mg/kg and 4 mg/kg of the subject's body weight.

84. The method of claim 83, wherein the LIV-1-ADC is administered at a dosage of 1.0 mg/kg of the subject's body weight or at a dosage of 1.25 mg/kg of the subject's body weight.

85. (canceled)

86. The method of claim 83 wherein the solid tumor is selected from the group consisting of non-small cell lung carcinoma (NSCLC) (squamous & non-squamous), small cell lung cancer, gastric/esophagogastric junction (GEJ) adenocarcinoma, esophageal squamous cell carcinoma, and head & neck squamous cell carcinoma.

87. A method of treating a solid tumor selected from the group consisting of non-small cell lung carcinoma (NSCLC) (squamous & non-squamous), small cell lung cancer, gastric/esophagogastric junction (GEJ) adenocarcinoma, esophageal squamous cell carcinoma, and head & neck squamous cell carcinoma comprising administering to the subject a LIV-1 antibody drug conjugate (LIV-1-ADC).

88. The method of claim 87 wherein the LIV-1-ADC is administered at a dosage between 1.0 mg/kg and 4 mg/kg of the subject's body weight.

89. The method of claim 87, wherein the LIV-1-ADC is administered at a dosage of 1.0 mg/kg of the subject's body weight or at a dosage of 1.25 mg/kg of the subject's body weight.

90. (canceled)

91. The method of claim 83, wherein the LIV-1-ADC is administered once weekly.

92-93. (canceled)

94. The method of claim 1, wherein treatment with LIV-1-ADC and a PD-1 antagonist increases levels of CD8+ T cells, dendritic cells, and/or macrophages in a tumor and/or tumor microenvironment.

95-97. (canceled)

98. The method of claim 1, wherein treatment with LIV-1-ADC and a PD-1 antagonist increases the expression of immune activation genes in cells of a tumor.

99. The method of claim 98, wherein immune activation genes are in cells selected from the group consisting of CD4+ T cells, CD8+ T cells, macrophages, and dendritic cells.

100. The method of claim 98, wherein the immune activation gene is an MHC gene, a cytokine gene, a chemokine gene, a lectin gene, SIGLEC1, MS4A4A, CD163, CXCL12, IL-18, and/or APOE.

101. The method of claim 99, wherein the immune activation genes are a HLA-DMA, HLA-DOA and IL-18.
